SEQUENCE LISTING (74 sequences)

SEQ ID NO: 1 - DNA, 417 bp, Acinetobacter sp. ADP1
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagc

SEQ ID NO: 2 - protein, 139 aa, Acinetobacter sp. ADP1
MLDAHISPEGTLSLQTIAMPADTNWSGDVFGGWIVSQMDLAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHAS

SEQ ID NO: 3 - DNA, 396 bp, Escherichia coli CFT073
atgtctacaacacataacgtccctcagggcgatcttgttttacgtactttagccatgcccgccgataccaatgccaatggtgacatctttggtggttggttaatgtcacaaatggatattggcggcgctattctggcgaaagaaattgcccacggtcgcgtagtgaccgtgcgggttgaaggaatgactttcttacgaccggttgcggtcggcgatgtggtgtgctgctatgcacgctgtgtccagaaagggacgacatcggttagcattaatattgaagtgtgggtgaaaaaagtcgcgtctgaacccatcgggcaacgctataaagcgacagaagcattatttaagtatgtcgcggttgatcctgaaggaaaacctcgcgccttacctgttgag

SEQ ID NO: 4 - protein, 132 aa, Escherichia coli CFT073
MSTTHNVPQGDLVLRTLAMPADTNANGDIFGGWLMSQMDIGGAILAKEIAHGRVVTVRVEGMTFLRPVAVGDVVCCYARCVQKGTTSVSINIEVWVKKVASEPIGQRYKATEALFKYVAVDPEGKPRALPVE

SEQ ID NO: 5 - DNA, 489 bp, Picrophilus torridus DSM 9790
atgaaagtcaaagatagcatggttgaaatcagtcgtctggttctgccggaagataccaatgtagttaacgcgttgtatggtggtcgtctggtcgaatggatggacaacatcgcaagcattacagcctacaaacatagccgtaagaacattgtgactggcagcatcgatagcctgttcttcatctctccaatccgtctgggcgacattgtgaccatccgctcatttgtgacctataccacccgcagtacgatggaaatcgagatcgatgtgtttagcgagaatgcgattaccggtgataagaagattactacacaggccttctttacctatgtggcaattgacgcggatggcaaaccggtggaaatcaaccagatcgaaccggagaatgacgaggagatgaaacgttacaaggaaggtgagattcgtagtgcggaacgtctgaaacgcctggccgaaaccaaagaacgtatcaaagcaaccttgaagatt

SEQ ID NO: 6 - protein, 163 aa, Picrophilus torridus DSM 9790
MKVKDSMVEISRLVLPEDTNVVNALYGGRLVEWMDNIASITAYKHSRKNIVTGSIDSLFFISPIRLGDIVTIRSFVTYTTRSTMEIEIDVFSENAITGDKKITTQAFFTYVAIDADGKPVEINQIEPENDEEMKRYKEGEIRSAERLKRLAETKERIKATLKI

SEQ ID NO: 7 - DNA, 39 bp, Artificial Sequence
  Oligonucleotide insert for upstream of 5′ start codon
acaatctagaggccagcctggccataaggagatactagt

SEQ ID NO: 8 - DNA, 36 bp, Artificial Sequence
  Oligonucleotide insert encoding six His tag upstream of stop codon
gccggcggccaaactggccaccatcaccatcaccat

SEQ ID NO: 9 - DNA, 453 bp, Artificial Sequence
  Codon-optimized version of thioesterase from Acinetobacter sp. ADP1
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 10 - protein, 151 aa, Artificial Sequence
  Thioesterase from Acinetobacter sp. ADP1 with His tag
MLDAHISPEGTLSLQTIAMPADTNWSGDVFGGWIVSQMDLAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 11 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggacagtgagccaaatggatctggccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 12 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 13 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctggttccgttgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 14 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCATISINQMTFLVPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 15 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatatggccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 16 - protein, 151 aa, Artificial Sequence
  Acinetobacter sp. ADP1 thioesterase variants
MLDAHISPEGTLSLQTIAMPADTNWSGDVFGGWIVSQMDMAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 17 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcgttggatgtgaaaggcaacaaacgtaccattgcggaagacctgaaacaacagttcctgcaacatgcaagcgccggcggccaaactggccaccatcaccatcaccat

SEQ ID NO: 18 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCATISINQMTFLVPVKVGDVISCYTKILKVGNTSIQMQIEVWDSHDSSRPPKRVTEGVFTFVALDVKGNKRTIAEDLKQQFLQHASAGGQTGHHHHHH

SEQ ID NO: 19 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 20 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCATISINQMTFLIPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 21 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgcaaccattagcatcaaccagatgaccttcctggttccgaggaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 22 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCATISINQMTFLVPRKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 23 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtgttaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 24 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCVTISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 25 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgttgtagtaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 26 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRCSTISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 27 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgtgttgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 28 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRVATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 29 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatattgccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 30 - protein, 151 aa, Artificial Sequence
  Acinetobacter sp. ADP1 thioesterase variants
MLDAHISPEGTLSLQTIAMPADTNWSGDVFGGWIVSQMDIAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 31 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgttgccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 32 - protein, 151 aa, Artificial Sequence
  Acinetobacter sp. ADP1 thioesterase variants
MLDAHISPEGTLSLQTIAMPADTNWSGDVFGGWIVSQMDVAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 33 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgcagccggtgcgattcatgcggaacgctttagcaaaggtcgttgtgcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 34 - protein, 151 aa, Artificial Sequence
  Acinetobacter sp. ADP1 thioesterase variants
MLDAHISPEGTLSLQTIAMPADTNWSGDVFGGWIVSQMDAAGAIHAERFSKGRCATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 35 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatctggccggtgcgattcatgcggaacgctttagcaaaggtcgtgcagcaaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 36 - protein, 151 aa, Artificial Sequence
  Acinetobacter sp. ADP1 thioesterase variants
MLDAHISPEGTLSLQTIAMPADTNWSGDVFGGWIVSQMDLAGAIHAERFSKGRAATISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 37 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtagcaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 38 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCSTISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 39 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatgcggccggtgcgattcatgcggaacgctttagcaaaggtcgttgtgttaccattagcatcaaccagatgaccttcctggttccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 40 - protein, 151 aa, Artificial Sequence
  Acinetobacter sp. ADP1 thioesterase variants
MLDAHISPEGTLSLQTIAMPADTNWSGDVFGGWTVSQMDAAGAIHAERFSKGRCVTISINQMTFLVPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 41 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtgttaccattagcatcaaccagatgaccttcctggttccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcgctggatgtgaaaggcaacaaacgtaccattgcggaagacctgaaacaacagttcctgcaacatgcaagcgccggcggccaaactggccaccatcaccatcaccat

SEQ ID NO: 42 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCVTISINQMTFLVPLKVGDVISCYTKILKVGNTSIQMQIEVWDSHDSSRPPKRVTEGVFTFVALDVKGNKRTIAEDLKQQFLQHASAGGQTGHHHHHH

SEQ ID NO: 43 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtagcaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 44 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCSTISINQMTFLIPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 45 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatgtggccggtgcgattcatgcggaacgctttagcaaaggtcgtgtggttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 46 - protein, 151 aa, Artificial Sequence
  Acinetobacter sp. ADP1 thioesterase variants
MLDAHISPEGTLSLQTIAMPADTNWSGDVFGGWTVSQMDVAGAIHAERFSKGRVVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 47 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtgttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 48 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 49 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatatggccggtgcgattcatgcggaacgctttagcaaaggtcgttgtgttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 50 - protein, 151 aa, Artificial Sequence
  Acinetobacter sp. ADP1 thioesterase variants
MLDAHISPEGTLSLQTIAMPADTNWSGDVFGGWTVSQMDMAGAIHAERFSKGRCVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 51 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatgcggccggtgcgattcatgcggaacgctttagcaaaggtcgttgtgttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 52 - protein, 151 aa, Artificial Sequence
  Acinetobacter sp. ADP1 thioesterase variants
MLDAHISPEGTLSLQTIAMPADTNWSGDVFGGWTVSQMDAAGAIHAERFSKGRCVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 53 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgcggccggtgcgattcatgcggaacgctttagcaaaggtcgtgcggttaccattagcatcaaccagatgaccttcctggttccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 54 - protein, 151 aa, Artificial Sequence
  Acinetobacter sp. ADP1 thioesterase variants
MLDAHISPEGTLSLQTIAMPADTNWSGDVFGGWIVSQMDAAGAIHAERFSKGRAVTISINQMTFLVPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 55 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgtggccggtgcgattcatgcggaacgctttagcaaaggtcgtgcggttaccattagcatcaaccagatgaccttcctggttccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 56 - protein, 151 aa, Artificial Sequence
  Acinetobacter sp. ADP1 thioesterase variants
MLDAHISPEGTLSLQTIAMPADTNWSGDVFGGWIVSQMDVAGAIHAERFSKGRAVTISINQMTFLVPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 57 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatatggccggtgcgattcatgcggaacgctttagcaaaggtcgtggggtcaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggtggatgtgaaaggcaacaaacgtaccattgcggaagacctgaaacaacagttcctgcaacatgcaagcgccggcggccaaactggccaccatcaccatcaccat

SEQ ID NO: 58 - protein, 151 aa, Artificial Sequence
  Acinetobacter sp. ADP1 thioesterase variants
MLDAHISPEGTLSLQTIAMPADTNWSGDVFGGWTVSQMDMAGAIHAERFSKGRGVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 59 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtgttaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 60 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCVTISINQMTFLIPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 61 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgtggccggtgcgattcatgcggaacgctttagcaaaggtcgtgcggttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 62 - protein, 151 aa, Artificial Sequence
  Acinetobacter sp. ADP1 thioesterase variants
MLDAHISPEGTLSLQTIAMPADTNWSGDVFGGWIVSQMDVAGAIHAERFSKGRAVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 63 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgcggccggtgcgattcatgcggaacgctttagcaaaggtcgtgcgagcaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 64 - protein, 151 aa, Artificial Sequence
  Acinetobacter sp. ADP1 thioesterase variants
MLDAHISPEGTLSLQTIAMPADTNWSGDVFGGWIVSQMDAAGAIHAERFSKGRASTISINQMTFLIPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 65 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgcggccggtgcgattcatgcggaacgctttagcaaaggtcgttgtgttaccattagcatcaaccagatgaccttcctgattccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 66 - protein, 151 aa, Artificial Sequence
  Acinetobacter sp. ADP1 thioesterase variants
MLDAHISPEGTLSLQTIAMPADTNWSGDVFGGWIVSQMDAAGAIHAERFSKGRCVTISINQMTFLIPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 67 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatctggccggtgcgattcatgcggaacgctttagcaaaggtcgttgtgttaccattagcatcaaccagatgaccttcctggttccgctgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 68 - protein, 151 aa, Artificial Sequence
  Acinetobacter sp. ADP1 thioesterase variants
MLDAHISPEGTLSLQTIAMPADTNWSGDVFGGWTVSQMDLAGAIHAERFSKGRCVTISINQMTFLVPLKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 69 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatatggccggtgcgattcatgcggaacgctttagcaaaggtcgtgcggttaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 70 - protein, 151 aa, Artificial Sequence
  Acinetobacter sp. ADP1 thioesterase variants
MLDAHISPEGTLSLQTIAMPADTNWSGDVFGGWTVSQMDMAGAIHAERFSKGRAVTISINQMTFLIPVKVGDVISCYTKILKVGNTSIQMQIEVWDSHDSSRPPKRVTEGVFTFVAVDVKGNKRTIAEDLKQQFLQHASAGGQTGHHHHHH

SEQ ID NO: 71 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggaccgtgagccaaatggatgcggccggtgcgattcatgcggaacgctttagcaaaggtcgttgtgttaccattagcatcaaccagatgaccttcctgattccggtgaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcgctggatgtgaaaggcaacaaacgtaccattgcggaagacctgaaacaacagttcctgcaacatgcaagcgccggcggccaaactggccaccatcaccatcaccat

SEQ ID NO: 72 - protein, 151 aa, Artificial Sequence
  Acinetobacter sp. ADP1 thioesterase variants
MLDAHISPEGTLSLQTIAMPADTNWSGDVFGGWTVSQMDAAGAIHAERFSKGRCVTISINQMTFLIPVKVGDVISCYTKILKVGNTSIQMQIEVWDSHDSSRPPKRVTEGVFTFVALDVKGNKRTIAEDLKQQFLQHASAGGQTGHHHHHH

SEQ ID NO: 73 - DNA, 453 bp, Artificial Sequence
  Acinetobacter sp. ADP1 thioesterase variants
atgctggatgcgcacatttcgccggaaggcaccctgagcctgcaaaccattgcaatgcccgccgataccaattggagtggtgatgtgttcggtggttggattgtgagccaaatggatgcggccggtgcgattcatgcggaacgctttagcaaaggtcgttgtgttaccattagcatcaaccagatgaccttcctgattccgcgtaaagttggtgatgtgattagctgctataccaagattctgaaggttggcaacaccagtattcagatgcagatcgaagtgtgggatagccatgatagcagtcgtccaccgaaacgcgttacggaaggcgtgtttacctttgttgcggttgatgtgaaaggcaacaaacgtaccattgcggaagacctgaaacaacagttcctgcaacatgcaagcgccggcggccaaactggccaccatcaccatcaccat

SEQ ID NO: 74 - protein, 151 aa, Artificial Sequence
  Acinetobacter sp. ADP1 thioesterase variants
MLDAHISPEGTLSLQTIAMPADTNWSGDVFGGWIVSQMDAAGAIHAERFSKGRCVTISINQMTFLIPRKVGDVISCYTKILKVGNTSIQMQIEVWDSHDSSRPPKRVTEGVFTFVAVDVKGNKRTIAEDLKQQFLQHASAGGQTGHHHHHH

(19) United States
(12) Patent Application Publication
(10) Pub. No.: US 2014/0342414 A1
(43) Pub. Date: Nov. 20, 2014

(54) DIRECT BIOCATALYTIC PRODUCTION OF ACRYLIC ACID AND OTHER CARBOXYLIC ACID COMPOUNDS

(71) Applicant: Codexis, Inc., Redwood City, CA (US)

(72) Inventors: Fernando Valle, Burlingame, CA (US); Nicholas John Agard, San Francisco, CA (US); Christopher Noriega, San Diego, CA (US)

(73) Assignee: Codexis, Inc., Redwood City, CA (US)

(21) Appl. No.: 14/345,495

(22) PCT Filed: Sep. 21, 2012
(86) PCT No.: PCT/US12/56639

§ 371 Date: Mar. 18, 2014

Related U.S. Application Data
(60) Provisional application No. 61/538,051, filed on Sep. 22, 2011.

(51) Int. Cl. C12P 7/40; C12N 9/16

(52) U.S. Cl. CPC C12P 7/40; C12N 9/16; C12Y 301/02
USPC 435/136; 435/254.2; 435/252.3; 435/254.11; 435/196

(57) ABSTRACT

The present disclosure relates to biocatalytic methods or processes for the synthesis of acrylic acid and its derivatives, or other carboxylic acid compounds of the formula R—CO2H, wherein R is a carbon chain of 5 carbons or fewer, such as methacrylic acid or 3-hydroxypropionic acid. More specifically, the disclosure relates to methods of using an acyl-CoA hydrolase (such as a thioesterase) as a biocatalyst for the hydrolysis (and removal of the CoA moiety) of a substrate acyl-CoA compound to produce the corresponding carboxylic acid compound, such as acrylic acid. In some embodiments, the disclosure provides non-naturally occurring microorganisms that have been transformed with a heterologous acyl-CoA hydrolase, such as a thioesterase, that is capable of hydrolyzing an acyl-CoA produced in a pathway of the microorganism and produce the corresponding carboxylic acid compound, thereby allowing methods for the direct fermentative production of the compound.

### 1. TECHNICAL FIELD

The present disclosure relates to biocatalytic methods or processes for the synthesis of acrylic acid and its derivatives, or other carboxylic acid compounds, such as methacrylic acid or 3-hydroxypropionic acid. More specifically, the disclosure relates to methods of using an acyl-CoA hydrolase (such as a thioesterase) as a biocatalyst for the hydrolysis (and removal of the CoA moiety) of a substrate acyl-CoA compound to produce the corresponding carboxylic acid compound, such as acrylic acid.

### 2. REFERENCE TO SEQUENCE LISTING

The official copy of the Sequence Listing is submitted concurrently with the specification as an ASCII formatted text file via EFS-Web, with a file name of “CX5-098WO2.txt”, a creation date of Sep. 17, 2012, and a size of 79,096 bytes. The Sequence Listing filed via EFS-Web is part of the specification and is incorporated in its entirety by reference herein.

### 3. BACKGROUND

Acrylic acid (also known as 2-propenoic acid, CH₂═CHCO₂H) is a carboxylic acid compound and widely used commodity chemical with annual world-wide production greater than 4 million metric tons. The major uses of acrylic acid and its salt, amide and ester derivatives are in the manufacture of polymeric products. The products derived from acrylic acid and its derivatives include for example, plastics, super-absorbent materials, exterior house paints, coatings for building materials, flocculants for waste water and treatment of sewage, printing inks, interior home applications, textile sizing, leather impregnation and finishing, masonry sealers, lacquers, and pharmaceutical binders. Currently most commercial production of acrylic acid is from propylene which is derived from petrochemical feedstock. This is an energy intense 2-step oxidation process. Instead of the well known chemical reactions, a more sustainable biocatalytic reaction would be highly desirable especially because it is anticipated that the current need for acrylic acid products will increase in the future. A number of recent publications describe more sustainable methods for producing acrylic acid, for example PCT patent publications WO2009/023039; WO2009/089457; WO2009/155382; WO2011/002892; WO2011/063363; WO2011/038364; WO2011/031083; and WO2011/011874; and US Patent Publications US2009/0275096 and US2010/0009419; however a need still exists for methods of producing acrylic acid which are efficient, sustainable and use less volatile non-petrochemical feedstocks. Like acrylic acid, the carboxylic acid compounds, methacrylic acid and 3-hydroxpropenoic acid (3HPA) are widely used commodity chemicals for which there is a need for sustainable methods of production using non-petrochemical feedstocks

### 4. SUMMARY

The present disclosure relates to recombinant polynucleotides, enzymes, recombinant host microorganisms, and associated biocatalytic methods for producing acrylic acid and/or related carboxylic acid compounds of the general formula R—CO₂H (or its unprotonated form), wherein R is a carbon chain of 5 carbons or fewer, including but not limited to, methacrylic acid, and 3-hydroxypropanoic acid (3HPA). The present disclosure is based in part on the discovery that certain microorganisms may be genetically manipulated to produce acrylic acid under certain culture conditions.

In some embodiments, the present disclosure provides, a non-naturally occurring (or recombinant) microorganism comprising: (a) a pathway that produces an acyl-CoA compound of formula R—(C═O)-CoA, wherein R is a carbon chain of 5 carbons or fewer; and (b) a heterologous polynucleotide encoding an acyl-CoA hydrolase capable of catalyzing the hydrolysis of the acyl-CoA compound, R—(C═O)-CoA, to the carboxylic acid compound, R—CO₂H. In some embodiments, the acyl-CoA compound of formula R—(C═O)-CoA is selected from: acrylyl-CoA, methacrylyl-CoA, and 3-hydroxypropionyl-CoA, and accordingly in some embodiments, the carboxylic acid compound, R—CO₂H is selected from: acrylic acid, methacrylic acid, and 3-hydroxypropionic acid (3HPA).

In some embodiments of the non-naturally occurring microorganism, the acyl-CoA hydrolase encoded by the heterologous polynucleotide is a thioesterase, optionally wherein the thioesterase is classified as a TE6 thioesterase, and optionally is derived from one of the following genes: Campylobacter jejuni (YP_{—}002344313.1); Haemophilus influenza (HIO827) (NP_{—}438987.1); Escherichia coli (AAN80186.1); Rattus norvegicus (EDM10006.1); Deinococcus geothermalis (YP_{—}605627.1); Picrophilus torridus DSM 9790 (YP_{—}023571.1); and Acinetobacter sp. ADP1 (YP_{—}047652.1, GI:50086142). In some embodiments, the acyl-CoA hydrolase is a thioesterase (TE) comprising an amino acid sequence having at least 80% identity to a sequence selected from SEQ ID NO: 2, 4, 6, and 10. In some embodiments, the TE is an engineered TE comprising an amino acid acid sequence having at least 80% identity to a reference sequence of SEQ ID NO: 2 or 10, and comprising at least one amino acid difference at a position relative to SEQ ID NO: 2 or 10 selected from I34, L40, C54, A55, V66, V68, and V117, and optionally wherein the amino acid differences are selected from I34T, L40A, L40I, L40M, L40V, C54A, C54V, A55S, A55V, V66I, V68L, V68R, and V117L.

In some embodiments, the present disclosure provides yeast cells, bacterial cells and fungal cells transformed with a heterologous polynucleotide sequence encoding an acrylyl-CoA hydrolase (such as a thioesterase enzyme) which is capable of catalyzing the conversion of acrylyl-CoA to acrylic acid in the host cell. In some embodiments the acrylic acid may be secreted from the host cell.

In one aspect, the disclosure relates to a method for making acrylic acid comprising reacting acrylyl-CoA in the presence of an acrylyl-CoA hydrolase to produce acrylic acid. In some embodiments of this aspect the method is conducted in vitro, in vivo or conducted partially in vivo and in vitro. In some embodiments of this aspect, the acrylyl-CoA hydrolase is a thioesterase.

In another aspect, the disclosure relates to the in vivo use or the in vitro use of an acrylyl-CoA hydrolase such as a thioesterase capable of hydrolyzing acryloyl-CoA to acrylic acid.

In a further aspect, the disclosure relates to a method for making acrylic acid comprising providing a microorganism transformed with at least one heterologous gene encoding an acrylyl-CoA hydrolase, and culturing the microorganism under sufficient culture conditions in the presence of a carbon source to promote the expression of the hydrolase and production of acrylic acid in the presence of the carbon source. In some embodiments, the microorganism is selected from the group of yeast; bacteria; or filamentous fungi such as but not limited to Bacillus, Lactobacillus, Escherichia, Rhizopus, Kluyveromyces, Myceliophthora, Rhodococcus, Trichoderma, Aspergillus, Saccharomyces, Pichia, Candida, Issatchenkia, or Yarrowia. In further embodiments, the method includes a recovery or isolating step. In other embodiments, the disclosure relates to the recombinant microorganisms comprising at least one heterologous gene encoding an acrylyl-CoA hydrolase that can used in the disclosed method.

In yet another aspect the disclosure relates to a method for producing acrylic acid comprising transforming a lactic acid producing microorganism with a heterologous polynucleotide encoding a thioesterase polypeptide, wherein the thioesterase polypeptide is capable of converting acrylyl-CoA to acrylic acid, culturing the transformed lactic acid producing microorganism in the presence of a carbon source and under sufficient conditions to produce acrylic acid and recovering the acrylic acid. In some embodiments of this aspect, the lactic acid producing microorganism further comprises at least one additional heterologous gene selected from a gene encoding a lactyl-CoA producing enzyme and an acrylyl-CoA producing enzyme.

In another aspect, the disclosure relates to a method for hydrolyzing acrylyl-CoA to acrylic acid or a derivative thereof comprising contacting an effective amount of a TE according to the invention with an acrylyl-CoA substrate for a period of time and under sufficient culture conditions to produce acrylic acid, wherein the TE is characterized by its ability to hydrolyze acrylyl-CoA to acrylic acid and wherein the acrylyl-CoA is produced from a cultured microbial cell. In some embodiments, the TE is a partially or substantially purified biologically derived TE.

In further aspects, the disclosure relates to a method for hydrolyzing acrylyl-CoA to acrylic acid or a derivative thereof comprising contacting an effective amount a thioesterase (TE) with an acrylyl-CoA substrate to for a period of time and under sufficient culture conditions to produce acrylic acid, wherein the TE is characterized by its ability to hydrolyze acrylyl-CoA to acrylic acid and wherein the TE is produced from a cultured microbial cell. In some embodiments the TE is a partially or substantially purified biologically derived TE.

In another aspect the disclosure relates to engineered TE polypeptides, the polynucleotides encoding them, and methods of using them, wherein the engineered TE polypeptides have improved characteristics as compared to a wild-type TE of SEQ ID NO: 2 (e.g., increased ability to hydrolyze acrylyl-CoA to acrylic acid), and wherein the improved characteristics are associated with residue differences as compared to SEQ ID NO:2 at residue positions I34, L40, C54, A55, V66, V68, and V117. In some embodiments, the engineered TE polypeptides comprise one or more of the amino acid residue differences: I34T, L40A, L40I, L40M, L40V, C54A, C54V, A55S, A55V, V66I, V68L, V68R, and V117L. In some embodiments, the engineered TE polypeptides are capable of hydrolyzing acrylyl-CoA to acrylic acid and comprise an amino acid sequence having at least 80% identity to a reference sequence selected from the even numbered sequences of SEQ ID NO: 12-74, and comprises one or more amino acid residue differences as compared to SEQ ID NO:2 or 10 at residue positions selected from: I34, L40, C54, A55, V66, V68, and V117.

There are additional features and embodiments of the invention described herein which will be apparent from the detailed description of the invention as provided below. The various embodiments described herein may be used in combination or separately.

### 5. BRIEF DESCRIPTION OF THE DRAWINGS

FIG. 1 depicts a plasmid map of pCK110900 as further described in Example 1. The lac promoter (lac), gene encoding for chloramphenicol acetyltransferase (cat), restriction sites SfiI, BglI, SpeI, XbaI and NgoMIV are indicated according to their respective locations on the plasmid.

FIG. 2 depicts an embodiment of a biosynthetic pathway in a recombinant microorganism for the direct production of acrylic acid where the pathway upstream of acrylyl-CoA involves production of lactic acid and lactyl-CoA, and/or production of propionyl-CoA.

FIG. 3 depicts an embodiment of a biosynthetic pathway in a recombinant microorganism for the direct production of acrylic acid where the pathway upstream of acrylyl-CoA involves production of β-alanine and β-alanyl-CoA.

FIG. 4A-C depict embodiments of biocatalytic hydrolysis reactions of acyl-CoA compounds to their corresponding carboxylic acid compounds by a thioesterase of the present disclosure: (A) hydrolysis of acrylyl-CoA to acrylic acid; (B) hydrolysis of methacrylyl-CoA to methacrylic acid; (C) hydrolysis of 3-hydroxypropionyl-CoA to 3-hydroxypropionic acid. FIG. 4D depicts an embodiment of a biocatalytic hydrolysis reaction of a generic acyl-CoA compound of formula R—(C═O)-CoA, wherein R is straight or branched carbon chain of 5 carbons or fewer, to its corresponding carboxylic acid by a thioesterase of the present disclosure.

### 6. DETAILED DESCRIPTION

### 6.1 Definitions

All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. It is intended that the present invention not be limited to the particular methodology, protocols, and reagents described herein, as these may vary, depending upon the context in which they are used.

Nonetheless, in order to facilitate understanding of the present invention, a number of terms are defined below.

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention that can be had by reference to the specification as a whole. Accordingly, the terms defined below are more fully defined by reference to the specification as a whole.

The phrase “hydrolyzing or catalyzing acrylyl CoA” means removal of the Coenzyme A (“CoA”) moiety from the substrate acrylyl-CoA by enzymatic conversion in the presence of an acrylyl-CoA hydrolase (such as for example a thioesterase).

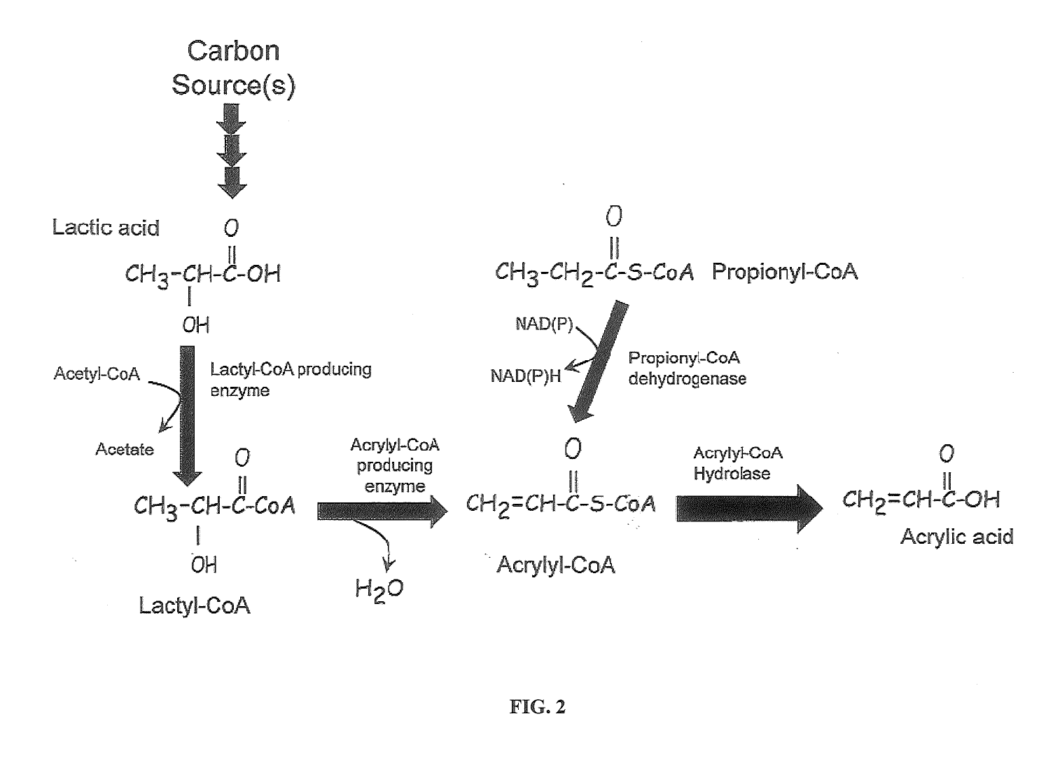

“Acrylyl-CoA,” “acrylic acid,” and “lactyl-CoA” each have the formula as illustrated in FIG. 2. The following terms are herein used synonymously “lactyl” and “lactoyl” and “acrylyl” and “acryloyl”. In addition, carboxylic acid products such as lactic acid or acrylic acid can be referred to interchangeably as the free acid (e.g., acrylic acid), its dissociated form (e.g., “acrylate”) or salts thereof.

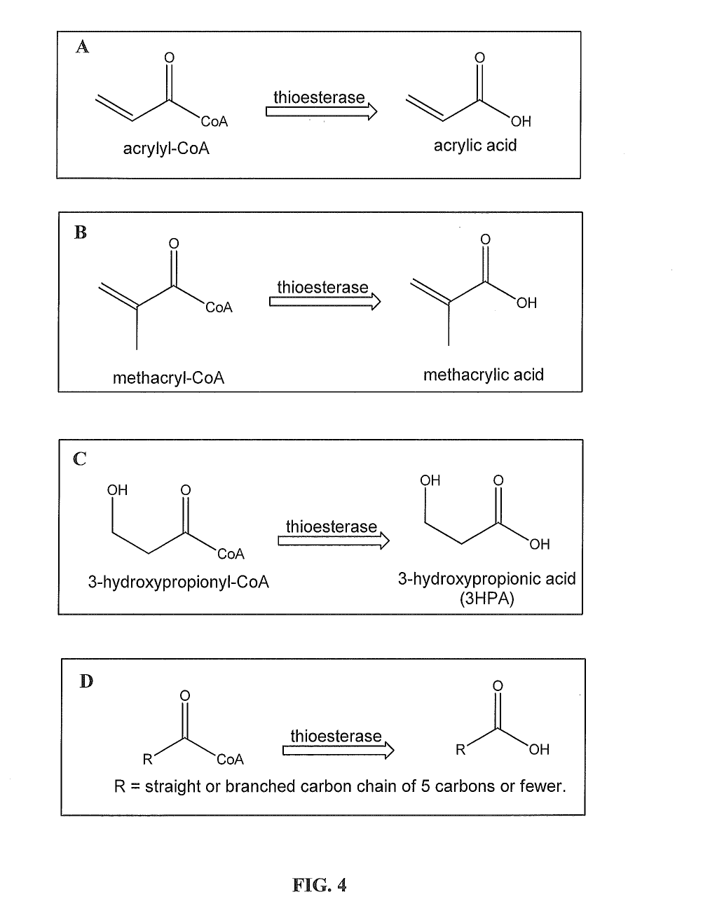

“Methacrylyl-CoA,” “methacrylic acid,” “3-hydroxypropionyl-CoA,” and “3-hydroxypropionic acid” (or “3HPA”) each have the formula as illustrated in FIG. 4.

“Acyl-CoA” as used herein refers to a compound comprising an acyl moiety attached through an acylthio bond to a CoA moiety. Exemplary acyl-CoA compounds include acrylyl-CoA, methacrylyl-CoA, and 3-hydroxypropoionyl-CoA. In embodiments, the present disclosure provides acyl-CoA compounds of formula R—(C═O)-CoA, wherein R is straight or branched carbon chain of 5 carbons or fewer, and the carbon chain can be substituted with functional groups selected from —F, —Cl, —Br, —I, —NH₂, —OH.

“Carboxylic acid” as used herein as used herein refers to any compound having a —CO₂H moiety (e.g., when protonated) or a —CO₂⁻ moiety (e.g., when unprotonated). Exemplary carboxylic acid compounds include acrylic acid, methacrylic acid, and 3-hydroyxypropionic acid. In embodiments, the present disclosure provides carboxylic acid compounds of formula R—CO₂H, wherein R is straight or branched carbon chain of 5 carbons or fewer, and the carbon chain can be substituted with functional groups selected from —F, —Cl, —Br, —I, —NH₂, —OH.

“Acrylyl-CoA hydrolases” as used herein are enzymes capable of hydrolyzing acrylyl-CoA to acrylic acid and CoA. Acrylyl-CoA hydrolases include esterases (capable of hydrolyzing acrylyl-CoA to acrylic acid and CoA) and thioesterases.

“Acyl-CoA hydrolases as used herein are enzymes capable of hydrolyzing an acyl-CoA compound to its corresponding carboxylic acid compound and CoA. Acyl-CoA hydrolases include esterases (capable of hydrolyzing acrylyl-CoA to acrylic acid and CoA) and thioesterases.

“Thioesterase(s)” (“TEs”) are identified as members of enzyme classification number EC 3.1.2.—wherein classification is based on the enzymes chemical reaction with a substrate. In addition, TEs are classified based on the ThYme database (Thioester-active enzyme; www.enzyme.cbirc.iastate.edu). Under this classification, TEs have been classified based on amino acid sequence similarity. The TEs are further divided into 24 different families (TE1-TE24). Reference is made to D. C. Cantu et al., (2011) Nucleic Acid Research 39:doi10:1093/nar/gkq1072. TEs according to the invention will have the ability to catalyze a thioester cleavage reaction hydrolyzing a thioester into an acid and a thiol.

As used herein a “short chain acrylyl-CoA hydrolase” means an acrylyl-CoA hydrolase having an amino acid sequence which is less than 300, less than 275, less than 250, less than 225, less than 200, less than 175, and also less than 150 amino acids.

The term “lactyl-CoA producing enzyme” means an enzyme capable of converting lactate to lactyl-CoA. A lactyl-CoA producing enzyme may be selected from transferases or synthetases such as for example, lactate-CoA transferases, coenzyme A transferases, propionate-CoA transferases, acetyl-CoA transferases, propionate-CoA:lactyl-CoA transferases, propionyl CoA:acetate CoA transferases, CoA synthetases, and further acyl activating enzymes, and short chain acyl-CoA synthetases. In one embodiment, the lactyl-CoA producing enzymes may be classified as E.C. 2.8.3.1. In another embodiment, the lactyl-CoA producing enzyme is a lactyl-CoA synthetase and may be classified as E.C. 6.2.1.1.

The term “acrylyl-CoA producing enzyme” means an enzyme capable of converting lactyl-CoA to acrylyl-CoA. An acrylyl-CoA producing enzyme may be selected from lactyl-CoA dehydratase, lactyl-coenzyme A dehydrase, lactoyl-coenzyme A dehydrase, acrylyl coenzyme A hydratase, and lactoyl-CoA hydrolyase. Acrylyl-CoA producing enzymes may be classified as E.C. 4.2.1.54.

The phrase “acrylic acid pathway” as used herein means the biotransformation of lactate to acrylate in a cell which includes the following enzymatic steps: a) the enzymatic conversion of lactic acid to lactyl-CoA by a lactyl-CoA producing enzyme; b) the enzymatic conversion of the lactyl-CoA produced in step a) to acrylyl-CoA by an acrylyl-CoA producing enzyme; and c) the enzymatic conversion of the acrylyl-CoA produced in step b) to acrylic acid by an acrylyl-CoA hydrolase enzyme.

The term “carbon source” refers to a substrate or compound suitable to be used as a source of carbon for prokaryotic or simple eukaryotic cell growth (e.g., yeast, bacterial or fungal) and/or suitable for end product production (such as the production of acrylic acid). Carbon sources can be in various forms including but not limited to carbohydrates, organic acids, alcohols, amino acids, and gases.

“Conversion” refers to the enzymatic conversion of a substrate to the corresponding product.

The term “in vivo” as used herein means that a process or reaction takes place inside a living intact cell or organism.

The term “in vitro” as used herein means that a process or reaction is carried out without cells (cell free) or in a substantially cell free environment comprising cells or cell components but in which the cells are no longer viable. A cell free system may include other additions such as additives (for example, co-factors such as but not limited to ATP, NAD(P), NADH and/or FAD).

“Naturally-occurring” or “wild-type” refers to the form found in nature. For example, a naturally occurring or wild-type polypeptide or polynucleotide sequence is a sequence present in an organism that can or could be isolated from a source in nature and which has not been intentionally modified by human manipulation. A wild-type organism or cell refers to an organism or cell that has not been intentionally modified by human manipulation.

“Recombinant” or “engineered” or “non-naturally occurring” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques.

“Recombinant microorganism” or “non-naturally occurring microorganism” refers to a cell or microorganism into which has been introduced a heterologous polynucleotide, gene, promoter, e.g., an expression vector, or to a cell or microorganism having a heterologous polynucleotide or gene integrated into the genome.

The term “expression” includes any step involved in the production of the polypeptide including, but not limited to, transcription, post-transcriptional modification, translation, post-translational modification, and secretion.

The term “culturing” refers to growing a population of microbial cells under suitable conditions in a liquid or solid medium. In particular embodiments, culturing refers to the fermentative bioconversion of a substrate to an end product. Fermentation can be aerobic, anaerobic or variations thereof.

The term “recoverable,” or “recovering” as used in reference to producing a composition (e.g., an acrylic acid composition) by a method of the present invention, refers to the harvesting, isolating, separating or collecting of a compound (e.g. acrylic acid) from a cell and/or culture medium.

“Isolated” with reference to a biological component (such as a polynucleotide or polypeptide) means that such component has been partially or completely separated from other biological components with which it is naturally associated with. For example, isolated polynucleotides or polypeptides include nucleic acid molecules and proteins purified by standard techniques.

The phrase “partially or substantially purified” when used in reference to a biologically derived TE means the TE is produced from a recombinant microorganism and is then separated from the microbial cells. The TE may be secreted into the cell culture and then removed by techniques know in the art or the cells may be disrupted. When desired the separation may include the removal of cell debris providing a cell free extract.

The terms “transform” or “transformation,” as used in reference to a cell, means a cell has a non-native nucleic acid sequence integrated into its genome or as an episome (e.g., plasmid) that is maintained through multiple generations.

The term “introduced,” as used in the context of inserting a nucleic acid sequence into a cell, means that the nucleic acid has been conjugated, transfected, transduced or transformed (collectively “transformed”) or otherwise incorporated into the genome of, or maintained as an episome in, the cell.

An “endogenous” polynucleotide, gene, promoter or polypeptide refers to any polynucleotide, gene, promoter or polypeptide that originates in a particular host cell. A polynucleotide, gene, promoter or polypeptide is not endogenous to a host cell if it has been removed from the host cell, subjected to laboratory manipulation, and then reintroduced into a host cell.

A “heterologous” polynucleotide, gene, promoter or polypeptide refers to any polynucleotide, gene, promoter or polypeptide that is introduced into a host cell that is not normally present in that cell, and includes any polynucleotide, gene, promoter or polypeptide that is removed from the host cell and then reintroduced into the host cell.

A polynucleotide or polypeptide that is “derived from” a particular organism refers to a wild-type polynucleotide or polypeptide that originates in the organism.

“Promoter sequence” is a nucleic acid sequence that is recognized by a host cell for expression of the coding region. The promoter sequence contains transcriptional control sequences, which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either endogenous or heterologous to the host cell. The promoter may also be homologous to the coding sequence to which it is operably linked. For example a polynucleotide construct comprising a nucleic acid molecule encoding an acrylyl-CoA hydrolase enzyme (such as a TE) may comprise a promoter that contains a sequence which is heterologous to the gene encoding the acrylyl-CoA hydrolase or may comprise a native acrylyl-CoA hydrolase promoter sequence. For purposes of this disclosure, a promoter is “heterologous” to a gene sequence if the promoter is not associated in nature with the gene.

“Operably linked” and “operably associated” are defined herein as a configuration in which a control sequence is appropriately placed at a position relative to the coding sequence of the DNA sequence such that the control sequence directs the expression of a polynucleotide and/or polypeptide.

“Percentage of sequence identity” and “percent identity” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which may also contain gaps to optimize the alignment) for alignment of the two sequences. The percentage may be calculated by determining the number of positions at which the identical nucleic acid base or amino acid residue occurs in both sequences to yield the number of matched positions, dividing the number of matched positions by the total number of positions in the window of comparison (including positions where one of the sequences has a gap(s) and multiplying the result by 100 to yield the percentage of sequence identity. Those of skill in the art appreciate that there are many established algorithms available to align two sequences and that different methods may give slightly different results. Alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). The Clustral (Chema R., Sugawara H., Koike T., Lopez R., Gibson T. J., Higgins D. G., Thompson J. D., (2003) Multiple sequence alignment with the Clustral series of programs, Nucleic Acids Res., 31, 3497-3500.) and T-Coffee (T-COFFEE: A novel method for multiple sequence alignments. Notredame, Higgins, Hering a, JMB 302 (205-217) 2000 software packages may also be used to align sequences. Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides over a “comparison window” to identify and compare local regions of sequence similarity.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, 150 or longer windows.

As used herein, “polynucleotide” refers to a polymer of deoxyribonucleotides or ribonucleotides in either single- or double-stranded form, and complements thereof.

The term “recombinant nucleic acid” has its conventional meaning. A recombinant nucleic acid, or equivalently, “polynucleotide,” is one that is inserted into a heterologous location such that it is not associated with nucleotide sequences that normally flank the nucleic acid as it is found in nature (for example, a nucleic acid inserted into a vector or a genome of a heterologous organism). Likewise, a nucleic acid sequence that does not appear in nature, for example a variant of a naturally occurring gene, is recombinant. A cell containing a recombinant nucleic acid, or protein expressed in vitro or in vivo from a recombinant nucleic acid are also “recombinant.” Examples of recombinant nucleic acids include a protein-encoding DNA sequence that is (i) operably linked to a heterologous promoter and/or (ii) encodes a fusion polypeptide with a protein sequence and a heterologous signal peptide sequence.

The term “expression vector” refers to a DNA molecule, linear or circular, that comprises a segment encoding a polypeptide of the invention, and which is operably linked to additional segments that provide for its transcription (e.g., a promoter, a transcription terminator sequence, enhancers) and optionally a selectable marker.

As used herein, the terms “peptide,” “polypeptide,” and “protein” are used interchangeably herein to refer to a polymer of amino acid residues. Amino acids are referred to herein by name, their commonly known three letter symbols or by the one-letter symbols recommended by the IUPAC-IUB Biochemical Nomenclature Commission. Nucleotides, likewise, may be referred to by their commonly accepted single-letter codes.

“Codon optimized” refers to changes in the codons of the polynucleotide encoding a protein to those preferentially used in a particular organism such that the encoded protein is efficiently expressed in the organism. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding enzymes may be codon optimized for optimal production from the host organism selected for expression.

“Preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See GCG Codon Preference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTs), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Conservative” amino acid substitutions or mutations refer to the interchangeability of residues having similar side chains, and thus typically involves substitution of the amino acid in the polypeptide with amino acids within the same or similar defined class of amino acids. However, as used herein, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. In some embodiments, conservatively substituted variations of the polypeptides of the present invention include substitutions of less than 10%, less than 5%, less than 2% and sometimes less than 1% of the amino acids of the polypeptide sequence, with a conservatively selected amino acid of the same conservative substitution group. Table 1 below shows exemplary conservative substitutions.

TABLE 1
Conservative Substitutions

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P, H | Other constrained (P, H) |
| N, Q, S, T | Other polar (N, Q, S, T) |
| Y, W, F | Other aromatic (Y, W, F) |
| C | None |

In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions.

“Non-conservative substitution” refers to substitution or mutation of an amino acid in the polypeptide with an amino acid with significantly differing side chain properties. Non-conservative substitutions may use amino acids between, rather than within, the defined groups listed above. In one embodiment, a non-conservative mutation affects (a) the structure of the peptide backbone in the area of the substitution (e.g., proline for glycine) (b) the charge or hydrophobicity, or (c) the bulk of the side chain.

“Control sequence” is defined herein to include all components, which are necessary or advantageous for the expression of a polypeptide of the present disclosure. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

### 6.2 Host Cells: Non-Naturally Occurring Microorganisms Comprising Heterologous Polynucleotides Encoding Acyl-CoA Hydrolases

The present disclosure provides non-naturally (or recombinant) microorganisms (as host cells) which comprise a biosynthetic pathway to an acyl-CoA compound and a heterologous polynucleotide encoding an enzyme having an acyl-CoA hydrolase activity (e.g., thioesterase (TE) activity) that hydrolyzes the acyl-thio bond of the acyl-CoA and thereby results in the production of the corresponding carboxylic acid compound (e.g., acrylic acid, methacrylic acid, or 3-hydroxypropionic acid) by the microorganism.

In some embodiments, the present disclosure contemplates that a non-naturally occurring microorganism useful for the direct production of a carboxylic acid compound of interest (e.g., acrylic acid, methacrylic acid, 3-hydroxypropionic acid) can be produced by heterologous transformation of a microorganism comprising a pathway that produces an acyl-CoA compound (e.g., acrylyl-CoA, methacryl-CoA, 3-hydroxyprionyl-CoA) for which hydrolysis of the acyl-thio bond results in the corresponding carboxylic acid product (e.g., acrylic acid, methacrylic acid, or 3-hydroxypropionic acid). More specifically, the microorganism is heterologously transformed with a polynucleotide encoding an enzyme having the appropriate acyl-CoA hydrolase activity to result in a recombinant microorganism capable of direct fermentative production of the carboxylic acid compound.

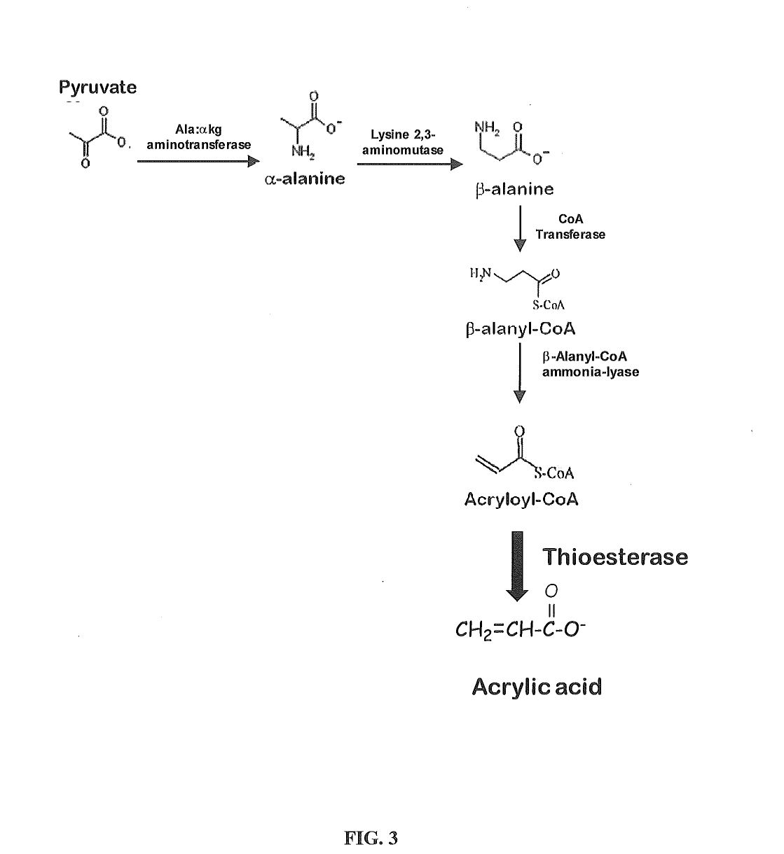

In one embodiment illustrated in FIG. 2, the non-naturally occurring microorganism has a biosynthetic pathway that produces the acyl-CoA compound, acrylyl-CoA, and is transformed with a a heterologous polynucleotide encoding an acrylyl-CoA hydrolase (e.g., a thioesterase as disclosed herein) that is capable of catalyzing the hydrolysis of acryl-CoA to acrylic acid. As further depicted in FIG. 2, in some embodiments the non-naturally occurring microorganism produces the acrylyl-CoA compound via one or more biosynthetic pathways that include the upstream compounds lactyl-CoA and/or propionyl-CoA. As depicted in FIG. 3, it also is contemplated that the non-naturally occurring microorganisms can comprises a biosynthetic pathway that produces β-alanine and β-alanyl-CoA upstream of acrylyl-CoA.

Biosynthetic pathways that can generate the acyl-CoA compounds 3-hydroxypropionyl-CoA or methacrylyl-CoA are known (see e.g., Henry et al., Biotechnolog. Bioeng. 2010, 106:462-473; Brunk et al., Biotechnol. Bioeng. 2012, 109:572-582; U.S. Pat. No. 8,076,120; U.S. Pat. Publ. 2010/0291644A1).

Accordingly, in some embodiments, the present disclosure provides a non-naturally occurring microorganism comprising a pathway that produces methacrylyl-CoA and further comprises a heterologous polynucleotide encoding methacryl-CoA hydrolase (e.g., an engineered thioesterase as disclosed herein) capable of hydrolyzing methacrylyl-CoA to methacrylic acid, thereby providing for direct fermentative production of the carboxylic acid compound, methacrylic acid.

In other embodiments, the present disclosure provides a non-naturally occurring microorganism comprising a metabolic pathway that produces 3-hydroxypropionyl-CoA which further comprises a heterologous polynucleotide encoding a 3-hydroxypropionyl-CoA hydrolase (e.g., an engineered thioesterase) capable of hydrolyzing 3-hydroxypropionyl-CoA to 3HPA, and thereby providing for direct fermentative production of the carboxylic acid compound, 3HPA.

In some embodiments a method for producing acrylic acid comprises culturing a non-naturally occurring microorganism capable of producing acrylyl-CoA comprising at least one heterologous polynucleotide that encodes an acrylyl-CoA hydrolase (such as a TE) expressed in a sufficient amount under sufficient culture conditions to produce acrylic acid from acrylyl-CoA. In some embodiments, the method for producing acrylic acid comprises culturing a non-naturally occurring microorganism that is capable of producing lactic acid and introducing at least one heterologous polynucleotide that encodes an acrylyl-CoA hydrolase (such as a TE) expressed in sufficient amounts under sufficient culture conditions to produce acrylic acid.

In some embodiments, it is contemplated that the non-naturally occurring microorganisms of the present disclosure can be obtained by heterologous transformation of a naturally-occurring microbial species that comprises a pathway resulting in an acyl-CoA compound of interest—e.g., a microorganism having a pathway that produces acrylyl-CoA, methacryl-CoA, or 3-hydroxypropionyl-CoA. In some embodiments, a non-naturally occurring microorganism (e.g., a recombinant host cell that already has been non-naturally modified by deletion of certain genes) that produces the acyl-CoA compound of interest can be heterologously transformed to provide a non-naturally occurring microorganism of the present disclosure. Generally, the present disclosure contemplates that any microbial species wherein the encoded gene product of the heterologous polynucleotide is capable of catalyzing the hydrolysis of the targeted acyl-CoA compound (e.g., acrylyl-CoA to acrylic acid) may be used as an exemplary microorganism. The microorganism may be a prokaryotic or eukaryotic microbial species including but not limited to yeast, filamentous fungi and bacteria.

In certain embodiments, the non-naturally occurring microorganism is a yeast. In various embodiments, the yeast is a species of Candida, Hansenula, Saccharomyces, Issatchenkia, Schizosaccharomyces, Pichia, Kluyveromyces, Torulaspora, Trichosporon, Yamadazyma, or Yarrowia. In various embodiments, the yeast is selected from the group consisting of Hansenula polymorpha, Saccharomyces cerevisiae, Saccharomyces carlsbergensis, Saccharomyces diastaticus, Saccharomyces norbensis, Saccharomyces kluyveri, Saccharomyces uvarum, Schizosaccharomyces pombe, Pichia pastoris, Pichia finlandica, Pichia trehalophila, Pichia ferniemtans, Issatchenkia orientalis, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Kluyveromyces thermotolerans, Kluyveromyces marxianus, Candida albicans, Candida krusei, Candida ethanolic, Candida revkaufi, Candida pulcherrima, Candida tropicalis, Candida utilis, Candida curvata, Candida diddensiae, Candida boldinii, Yarrowia lipolytica, Yarrowia stipitis, and Yarrowia paralipolytica and synonyms or taxonomic equivalents thereof.

In some embodiments, the yeast is a recombinant yeast that has for example been modified to include heterologous polynucleotides other than an exogenous polynucleotide encoding an acyl-CoA hydrolase (e.g., acrylyl-CoA hydrolase) according to the disclosure. Recombinant or modified yeast can be found in the Open Biosystems collection found at the website www.openbiosystems.com/GeneExpression/Yeast/YKO/. (See e.g., Winzeler et al. (1999) Science 285:901-906). In some embodiments, the recombinant yeast will include 1 or more (such as 2, 3, 4, 5, or more) additional heterologous polynucleotides encoding enzymes other than the acrylyl-CoA hydrolase.

In some embodiments, the non-naturally occurring microorganism is a bacterium. Suitable prokaryotic cells include Gram-positive, Gram-negative and Gram-variable bacterial cells. Examples of bacterial host cells include Bacillus (such as B. subtilis, B. licheniformis, B. megaterium, B. stearothermophilus and B. amyloliquefaciens), Streptomyces (such as S. ambofaciens, S. achromogenes, S. avermitilis, S. coelicolor, S. aureofaciens, S. aureus, S. fungicidicus, S. griseus, and S. lividans), Saccharophagus (such as S. degradans) and Streptococcus (such as S. equisimiles, S. pyogenes, and S. uberis) species.

Exemplary bacteria also include species selected from Escherichia coli, Klebsiellla (e.g., K. oxytoca), Acetobacter, Actinobacillus succinogenes, Mannheimia succiniciproducens, Rhizobium etli, Corynebacterium glutamicum, Gluconobacter oxydans, Zymomonas mobilis, Lactococcus lactis, Lactobacillus (e.g., L. plantarum and L. lactis), Clostridium (e.g., C. acetobutylicum, propionicum and tyrobutyricum), Pseudomonas fluorescens, and Pseudomonas putida.

In some embodiments, the recombinant bacteria have been modified to include heterologous polynucleotides other than the heterologous polynucleotide encoding an acrylyl-CoA hydrolase and these recombinant microorganisms will include 1 or more (such as 2, 3, 4, 5, or more) additional heterologous polynucleotides encoding enzymes other than the acrylyl-CoA hydrolase.

Suitable fungi including species selected from but are not limited to Ascomycota, Basidiomycota, Deuteromycota, Zygomycota, Fungi imperfecti. In some embodiments, fungal host cells are filamentous fungal cells, including all filamentous forms of the subdivision Eumycotina and Oomycota. Hawksworth et al., In Ainsworth and Bisby's DICTIONARY OF THE FUNGI, 8^{th} edition, 1995, CAB International, University Press, Cambridge, UK. Filamentous fungi are characterized by a vegetative mycelium with a cell wall composed of chitin, cellulose and other complex polysaccharides, and are morphologically distinct from yeast. In some embodiments, the host cell may be a species of Acremonium, Aspergillus, Chrysosporium, Fusarium, Gibberella, Humicola, Hypocrea, Mucor, Myceliophthora, Neurospora, Piromyces, Podospora, Rhizobium, Rhizomucor, Rhizopus, Sporotrichum, Talaromyces, Thermoascus, Thermotoga, Thielavia, Trichoderma, or corresponding teleomorphs, or anamorphs, and synonyms or taxonomic equivalents thereof. In some embodiments, the Trichoderma species may be T. longibrachiatum, T. viride, Hypocrea jecorina or T. reesei, T. koningii, and T. harzianum. In some embodiments, the Aspergillus species may be A. terreus, A. awamori, A. fumigatus, A. japonicus, A. nidulans, A. niger, A. aculeatus, A. foetidus, A. oryzae, A. sojae, and A. kawachi. In some embodiments the Fusarium species may be F. bactridioides, F. cerealis, F. crookwellense, F. culmorum, F. graminearum, F. graminum, F. oxysporum, F. roseum, and F. venenatum. In some embodiments the Neurospora species may be N. crassa. In some embodiments the Humicola species may be H. insolens, H. grisea, and H. lanuginosa. In some embodiments the Rhizopus species may be R. oryzae and R. niveus.

In some embodiments, the recombinant filamentous fungal microorganisms have been modified to include heterologous polynucleotides other than the heterologous polynucleotide encoding an acrylyl-CoA hydrolase and these recombinant microorganisms will include 1 or more (such as 2, 3, 4, 5, or more) additional heterologous polynucleotides encoding enzymes other than the acrylyl-CoA hydrolase.

In some embodiments the microorganism is an E. coli, Lactobacillus sp., Clostridium sp., Yarrowia sp., Rhizopus sp., Saccharomyces sp., Saccharophagus sp., Myceliophthora sp., Issatchenkia sp., or Kluyveromyces sp.

Strains that may be used in the practice of the invention (both prokaryotic and eukaryotic strains) may be obtained from any suitable source, including but not limited to the American Type Culture Collection (ATCC), or other biological depositories such as Deutsche Sammlung von Mikroorganismen and Zellkulturen GmbH (DSM), Centraalbureau Voor Schimmelcultures (CBS), and the Agricultural Research Service Patent Culture Collection, Northern Regional Research Center (NRRL).

### 6.3 DNA Constructs and Heterologous Polynucleotides

A microorganism according to the disclosure capable of producing acrylyl-CoA and/or capable of producing lactic acid will be engineered to comprise a heterologous polynucleotide encoding an acrylyl-CoA hydrolase that is capable of converting acrylyl-CoA to acrylic acid. In some embodiments, the acrylyl-CoA hydrolase will be a thioesterase (TE). In some embodiments the TE will be a TE classified as EC 3.1.2.* (wherein * denotes any number at this position) and in some embodiments the TE will be classified as EC3.1.2.14. In some embodiments, the polynucleotide encoding an acrylyl-CoA hydrolase according to the invention will be a codon optimized polynucleotide.

In some embodiments the TEs useful in the methods according to the invention will be plant, bacterial, animal, yeast or fungal derived TEs and reference is made to PCT publication No. WO2010/075483 which includes a long list of source organisms and thioesterase enzymes.

In some embodiments, the TE is a plant derived TE, for example, the genes fatA, fatB, fatB2, fat B3 and tesA which encode TE. These genes may be derived from but are not limited to the following source organisms: Arabidopsis, Cinnamonum, Cuphea, Glycine and Umbellularia. In addition exemplary GenBank Accession numbers are Z36912; Z36911, X73849, U17098, U17076 and M94159 and reference is made to A. Jones et al., (1995) The Plant Cell, Vol. 7:359-371.

In some embodiments, the TE will be a thioesterase classified in family TE1-TE24 of the ThYme database classification. In some embodiments, the TE will comprise a TE classified in family TE2, TE 4, TE 6, TE8, TE9, TE10, TE11, TE13, TE18, or TE24. In some embodiments the TE will be any TE as described in Table 2. In some embodiments, the TE will be classified as a TE6 according to the ThYme classification system.

In some embodiments, the TE will be derived from an Acinetobacter sp., an E. coli sp., or a Picrophilus sp.

In some embodiments, the TE will be encoded by a polynucleotide having at least 75%, at least 80%, at least 85%, at least 88%, at least 90%, at least 91%, at least 92%, at least 93%, at least 94%, at least 95%, at least 96%, at least 97%, at least 98%, at least 99% and even 100% sequence identity to the polynucleotide of SEQ ID NO: 1 (Genbank Accession No. YP_{—}047652; GI:50086142).

(SEQ ID NO: 1)
ATGCTGGATGCGCACATTTCGCCGGAAGGCACCCTGAG
CCTGCAAACCATTGCAATGCCCGCCGATACCAATTGGA
GTGGTGATGTGTTCGGTGGTTGGATTGTGAGCCAAATG
GATCTGGCCGGTGCGATTCATGCGGAACGCTTTAGCAA
AGGTCGTTGTGCAACCATTAGCATCAACCAGATGACCT
TCCTGGTTCCGGTGAAAGTTGGTGATGTGATTAGCTGC
TATACCAAGATTCTGAAGGTTGGCAACACCAGTATTCA
GATGCAGATCGAAGTGTGGGATAGCCATGATAGCAGTC
GTCCACCGAAACGCGTTACGGAAGGCGTGTTTACCTTT
GTTGCGGTTGATGTGAAAGGCAACAAACGTACCATTGC
GGAAGACCTGAAACAACAGTTCCTGCAACATGCAAGC.

In some embodiments the polynucleotide encoding the TE is a codon optimized version of the polynucleotide of polynucleotide of SEQ ID NO: 1.

In some embodiments, the TE comprises an amino acid sequence having at least 85%, at least 88%, at least 90%, at least 91%, at least 92%, at least 93%, at least 94%, at least 95%, at least 96%, at least 97%, at least 98%, at least 99% and even 100% sequence identity to SEQ ID NO: 2.

(SEQ ID NO: 2)
MLDAHISPEGTLSLQTIAMPADTNWSGDVFGGWIVSQMDL
AGAIHAERFSKGRCATISINQMTFLVPVKVGDVISCYTKI
LKVGNTSIQMQIEVWDSHDSSRPPKRVTEGVFTFVAVDVK
GNKRTIAEDLKQQFLQHAS

In some embodiments the TE will be coded for by a polynucleotide having at least at least 75%, at least 70%, at least 85%, at least 88%, at least 90%, at least 91%, at least 92%, at least 93%, at least 94%, at least 95%, at least 96%, at least 97%, at least 98%, at least 99% and even 100% sequence identity to the polynucleotide of SEQ ID NO: 3 (Genbank Accession No.: AAN80186.1).

(SEQ ID NO: 3)
ATGTCTACAACACATAACGTCCCTCAGGGCGATCTTGTTTT
ACGTACTTTAGCCATGCCCGCCGATACCAATGCCAATGGTG
ACATCTTTGGTGGTTGGTTAATGTCACAAATGGATATTGGC
GGCGCTATTCTGGCGAAAGAAATTGCCCACGGTCGCGTAGT
GACCGTGCGGGTTGAAGGAATGACTTTCTTACGACCGGTTG
CGGTCGGCGATGTGGTGTGCTGCTATGCACGCTGTGTCCAG
AAAGGGACGACATCGGTTAGCATTAATATTGAAGTGTGGGT
GAAAAAAGTCGCGTCTGAACCCATCGGGCAACGCTATAAAG
CGACAGAAGCATTATTTAAGTATGTCGCGGTTGATCCTGAA
GGAAAACCTCGCGCCTTACCTGTTGAG

In some embodiments the polynucleotide encoding the TE is a codon optimized version of the polynucleotide of SEQ ID NO: 3.

In some embodiments, the TE comprises an amino acid sequence having at least 85%, at least 88%, at least 90%, at least 91%, at least 92%, at least 93%, at least 94%, at least 95%, at least 96%, at least 97%, at least 98%, at least 99% and even 100% sequence identity to SEQ ID NO: 4.

(SEQ ID NO: 4)
MSTTHNVPQGDLVLRTLAMPADTNANGDIFGGWLMSQMDIGG
AILAKEIAHGRVVTVRVEGMTFLRPVAVGDVVCCYARCVQKG
TTSVSINIEVWVKKVASEPIGQRYKATEALFKYVAVDPEGKP
RALPVE

In some embodiments, the TE will be coded for by a polynucleotide having at least 75%, at least 80%, at least 85%, at least 88%, at least 90%, at least 91%, at least 92%, at least 93%, at least 94%, at least 95%, at least 96%, at least 97%, at least 98%, at least 99% and even 100% sequence identity to the polynucleotide of SEQ ID NO: 5 (Genbank Accession No.: YP_{—}023571.1).

(SEQ ID NO: 5)
ATGAAAGTCAAAGATAGCATGGTTGAAATCAGTCGTCTGGTT
CTGCCGGAAGATACCAATGTAGTTAACGCGTTGTATGGTGGT
CGTCTGGTCGAATGGATGGACAACATCGCAAGCATTACAGCC
TACAAACATAGCCGTAAGAACATTGTGACTGGCAGCATCGAT
AGCCTGTTCTTCATCTCTCCAATCCGTCTGGGCGACATTGTG
ACCATCCGCTCATTTGTGACCTATACCACCCGCAGTACGATG
GAAATCGAGATCGATGTGTTTAGCGAGAATGCGATTACCGGT
GATAAGAAGATTACTACACAGGCCTTCTTTACCTATGTGGCA
ATTGACGCGGATGGCAAACCGGTGGAAATCAACCAGATCGAA
CCGGAGAATGACGAGGAGATGAAACGTTACAAGGAAGGTGAG
ATTCGTAGTGCGGAACGTCTGAAACGCCTGGCCGAAACCAAA
GAACGTATCAAAGCAACCTTGAAGATT

In some embodiments, the polynucleotide encoding the TE is a codon optimized version of the polynucleotide of SEQ ID NO: 5.

In some embodiments, the TE comprises an amino acid sequence having at least 85%, at least 88%, at least 90%, at least 91%, at least 92%, at least 93%, at least 94%, at least 95%, at least 96%, at least 97%, at least 98%, at least 99% and even 100% sequence identity to SEQ ID NO: 6.

(SEQ ID NO: 6)
MKVKDSMVEISRLVLPEDTNVVNALYGGRLVEWMDNIASITA
YKHSRKNIVTGSIDSLFFISPIRLGDIVTIRSFVTYTTRSTM
EIEIDVFSENAITGDKKITTQAFFTYVAIDADGKPVEINQIE
PENDEEMKRYKEGEIRSAERLKRLAETKERIKATLKI

However, the invention is not limited to the above exemplified TEs. Genes encoding TEs having the capacity to hydrolyze acrylyl-CoA to acrylic acid would be routine to screen and these genes from similar organisms or unrelated organisms as described above may be used in the methods of the invention.

In some embodiments, the TE will be a wild-type TE and in other embodiments the TE will be a mutant or an engineered variant of a wild-type TE (e.g., wild-type TE polypeptides of SEQ ID NO: 2, 4, and 6).

In the embodiments, the mutant or engineered variants of a wild-type TE and corresponding polynucleotides encoding such engineered TE can be obtained using methods used by those skilled in the art. The engineered TE described herein can be obtained by subjecting the naturally occurring polynucleotide encoding the naturally occurring TE (e.g., TE polypeptides of SEQ ID NO: 2, 4, and 6) or a previously engineered TE (e.g., engineered TE polypeptides of even-numbered SEQ ID NO: 12-74) to mutagenesis and/or directed evolution methods, as described herein (see e.g., below and in Example 3).

Exemplary directed evolution techniques include mutagenesis and/or DNA shuffling as described in Stemmer, 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, Nat. Biotechnol. 16:258-261), mutagenic PCR (Caldwell et al., 1994, PCR Methods Appl. 3:S136-S140), and cassette mutagenesis (Black et al., 1996, Proc Natl Acad Sci USA 93:3525-3529). Mutagenesis and directed evolution techniques useful for the purposes herein are also described in the following references: Ling, et al., 1997, Anal. Biochem. 254(2):157-78; Dale et al., 1996, “Oligonucleotide-directed random mutagenesis using the phosphorothioate method,” In Methods Mol. Biol. 57:369-74; Smith, 1985, Ann. Rev. Genet. 19:423-462; Botstein et al., 1985, Science 229:1193-1201; Carter, 1986, Biochem. J. 237:1-7; Kramer et al., 1984, Cell, 38:879-887; Wells et al., 1985, Gene 34:315-323; Minshull et al., 1999, Curr Opin Chem Biol 3:284-290; Christians et al., 1999, Nature Biotech 17:259-264; Crameri et al., 1998, Nature 391:288-291; Crameri et al., 1997, Nature Biotech 15:436-438; Zhang et al., 1997, Proc Natl Acad Sci USA 94:45-4-4509; Crameri et al., 1996, Nature Biotech 14:315-319; Stemmer, 1994, Nature 370:389-391; Stemmer, 1994, Proc Natl Acad Sci USA 91:10747-10751; WO 95/22625; WO 97/0078; WO 97/35966; WO 98/27230; WO 00/42651; WO 01/75767 and U.S. Pat. No. 6,537,746. All publications are incorporated herein by reference.

In some embodiments, the present disclosure provides an engineered thioesterase TE polypeptide capable of hydrolyzing acrylyl-CoA to acrylic acid, wherein the engineered TE polypeptide is derived by directed evolution of a wild-type TE classified in family TE2, TE 4, TE 6, TE8, TE9, TE10, TE11, TE13, TE18, or TE24. In some embodiments, the engineered TE can be derived from a wild-type TE classified as a TE6 according to the ThYme classification system. In some embodiments, the engineered TE polypeptide can be derived from a wild-type TE polypeptide from a microorganism selected from Acinetobacter sp., E. coli, and Picrophilus sp. In some embodiments, the engineered TE polypeptide can be derived from a wild-type TE polypeptide having an amino acid sequence comprising any one of SEQ ID NO: 2, 4, or 6. Accordingly, the engineered TE polypeptide can be derived of a directed evolution of a polynucleotide encoding a wild-type TE polypeptide having an amino acid sequence comprising any one of SEQ ID NO: 2, 4, or 6. Such polynucleotides encoding an amino acid sequence comprising any one of SEQ ID NO: 2, 4, or 6, can be selected from the polynucleotide sequences of SEQ ID NO: 2, 4, 6, and 10.

In some embodiments, the engineered TE has improved characteristics relative to a wild-type TE from which it is derived by directed evolution, for example an improved ability of hydrolyzing an acyl-CoA compound (e.g., acrylyl-CoA) to its corresponding carboxylic acid product (e.g., acrylic acid). Exemplary engineered TE polypeptides having improved characteristics relative to the wild-type TE of SEQ ID NO: 2 (or 10) are provided herein as the polypeptides of even-numbered SEQ ID NO: 12-74 (see Table 4, Example 3 and Sequence Listing). From an analysis of the exemplary polypeptides, the improved characteristics (e.g., increased ability to hydrolyze acrylyl-CoA to acrylic acid) are associated with residue differences as compared to SEQ ID NO:2 at residue positions I34, L40, C54, A55, V66, V68, and V117. The specific amino acid residue differences at each of these positions that are associated with the improved properties include: I34T, L40A, L40I, L40M, L40V, C54A, C54V, A55S, A55V, V66I, V68L, V68R, and V117L.

In light of the guidance provided herein, it is further contemplated that any of the exemplary engineered TE polypeptides of even numbered SEQ ID NO: 12-74 can be used as the starting amino acid sequence for synthesizing other engineered TE polypeptides, for example by subsequent rounds of evolution that incorporate new combinations of the various amino acid differences from other exemplary engineered TE polypeptides provided in Table 4 (of Example 3) and other residue positions described herein. Further improvements may be generated by including amino acid differences at residue positions that had been maintained as unchanged throughout earlier rounds of evolution.

Accordingly, in some embodiments, the present disclosure provides an engineered TE polypeptide capable of hydrolyzing acrylyl-CoA to acrylic acid which comprises an amino acid sequence having at least 80%, 85%, 86%, 87%, 88%, 89%, 90%, 91%, 92%, 93%, 94%, 95%, 96%, 97%, 98%, 99% or more sequence identity to a reference sequence of SEQ ID NO:2 or 10 and one or more amino acid residue differences as compared to SEQ ID NO:2 or 10 at residue positions selected from: I34, L40, C54, A55, V66, V68, and V117.

In some embodiments, the engineered TE polypeptide is capable of hydrolyzing acrylyl-CoA to acrylic acid with improved properties as compared to the reference polypeptide of SEQ ID NO:2 or 10, and comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from the even numbered sequences of SEQ ID NO: 12-74, and comprises one or more residue differences as compared to SEQ ID NO:2 or 10 at residue positions selected from: I34, L40, C54, A55, V66, V68, and V117. In some embodiments, the reference amino acid sequence is selected from SEQ ID NO: 24, 40, 52, 66, and 74. In some embodiments, the reference amino acid sequence is SEQ ID NO: 24. In some embodiments, the reference amino acid sequence is SEQ ID NO: 74.

In some embodiments, the engineered proline hydroxylase polypeptide comprises an amino acid sequence having at least a combination of residues differences as compared to SEQ ID NO: 2 or 10 selected from the combinations of residue differences relative to SEQ ID NO: 2 or 10 present in the polypeptides of even numbered SEQ ID NO: 34-74 (see Table 4 and Sequence Listing). The combinations of residue differences can be selected of even numbered SEQ ID NO: 34-74 include the following; (a) I34T, A55S; (b) A55V, I34T, L40A, V68L; (c) A55V, I34T, V68L, V117L; (d) I34T, A55S, V66I; (e) A55V, I34T, L40V, C54V, V66I, V68L; (f) A55V, I34T, V66I, V68L; (g) A55V, I34T, L40M, V66I, V68L; (h) A55V, I34T, L40A, V66I, V68L; (i) A55V, L40A, C54A; (j) A55V, L40V, C54A, V68L; (k) A55V, I34T, L40M, C54G, V66I, V68L; (l) A55V, I34T, V66I; (m) A55V, L40V, C54A, V66I, V68L; (n) L40A, C54A, A55S, V66I; (o) A55V, L40A, V66I, V68L; (p) A55V, I34T, V68L; (q) A55V, I34T, L40M, C54A, V66I; (r) A55V, I34T, L40A, V66I, V117L; and (s) A55V, L40A, V66I; V68R.

As will be appreciated by the skilled artisan, in some embodiments, one or a combination of residue differences above that is selected can be kept constant (i.e., maintained) in the engineered TE polypeptide as a core feature, and additional residue differences at other residue positions incorporated into the sequence to generate additional engineered TE polypeptides with improved properties. Accordingly, it is to be understood for any engineered TE containing one or a subset of the residue differences above, the present disclosure contemplates other engineered TE polypeptides that comprise the one or subset of the residue differences, and additionally one or more residue differences at the other residue positions disclosed herein. By way of example and not limitation, an engineered TE comprising a residue difference at residue position A55, can further incorporate one or more residue differences at the other residue positions, e.g., I34, L40, C54, V66, V68, and V117. Another example is an engineered TE comprising a residue difference at residue position V68, which can further comprise one or more residue differences at the other residue positions, e.g., I34, L40, C54, A55, V66, and V117.

Because of the knowledge of the codons corresponding to the various amino acids, availability of a polypeptide sequence provides a description of all the polynucleotides capable of encoding the subject polypeptides disclosed herein. The degeneracy of the genetic code, where the same amino acids are encoded by alternative or synonymous codons allows an extremely large number of nucleic acids to be made, all of which encode a TE disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein (e.g., the TE polypeptides having amino acid sequences of even numbered SEQ ID NO: 2-74).

In one embodiment, the present disclosure provides a polynucleotide encoding a TE polypeptide, wherein the polynucleotide comprises a nucleotide sequence having at least 70%, 75%, 80%, 85%, 86%, 87%, 88%, 89%, 90%, 91%, 92%, 93%, 94%, 95%, 96%, 97%, 98%, 99% or more identity to a reference sequence selected from SEQ ID NO: 1, 3, 5, and 9. In some embodiments, the polynucleotide encodes an engineered TE polypeptide capable of hydrolyzing acrylyl-CoA to acrylic acid, wherein the polynucleotide comprises a nucleotide sequence having at least 70%, 75%, 80%, 85%, 86%, 87%, 88%, 89%, 90%, 91%, 92%, 93%, 94%, 95%, 96%, 97%, 98%, 99% or more identity to a reference sequence selected from of an odd-numbered SEQ ID NO: 11-73.

Moreover, exemplary methods for testing the conversion of acrylyl-CoA to acrylic acid in a non-naturally occurring microorganism can be performed by detection methods well known in the art. For example, reference is made to Sambrook et al., MOLECULAR CLONING: A LABORATORY MANUAL, 3^{rd} Ed., Cold Spring Harbor Laboratory, NY (2001) and CURRENT PROTOCOLS IN MOLECULAR BIOLOGY, F. M. Ausubel et al., eds., Current Protocols (as supplemented through 2009).

In some embodiments, the TE is selective against lactyl-CoA. The term “selective against lactyl-CoA” refers to an enzyme that prefers acrylyl-CoA as a substrate compared to lactyl-CoA. For example when both acrylyl-CoA and lactyl-CoA are available as a substrate in equal amounts, in concentrations between about 10 μM to 10 mM, the ratio of the initial rate of cleavage for acrylyl-CoA as opposed to lactyl-CoA is greater than 1.0 (and in some embodiments great than 1.5, greater than 2, greater than 3, greater than 4, greater than 5, greater than 10, greater than 15, greater than 20, greater than 30, greater than 50, greater than 100 or even greater than 200.

The present disclosure also contemplates thioesterases (both naturally occurring and engineered) having the capacity to hydrolyze substrates other than acrylyl-CoA to commercially relevant carboxylic acid products other than acrylic acid. Thioesterases can exhibit a wide range of substrate specificities (see e.g., as discussed in: Jung et al., BMC Biochemistry, 2011, 12: 1-14; and Lee et al., Biocat. Agric. Biotechnol. 2012, 1: 95-104). For example, the E. coli thioesterase TesA can hydrolyze efficiently thioesters, aromatic amino-acid-derived esters, p-nitrophenyl esters, triglycerides and lysophosphatidyl choline esters (Lee et al., ibid). In another example, the E. coli thioesterase II (TesB) was reported to have a broad specificity for catalyzing the conversion of acyl-CoA compounds having C₆-C₁₈ chain length to their corresponding free fatty acids. It also has been reported that the thioesterase TesB can produce R-3-hydroxybutyric acid indicating that it can convert an hydroxyl-C₄-CoA substrate (Liu et al., Appl. Microbiol. Biotechnol. 2007, 76: 811-818).

Accordingly, in some embodiments, the present disclosure contemplates a thioesterase (including engineered variants of the Acinetobacter sp. ADP1 thioesterase of SEQ ID NO: 2) capable of hydrolyzing an acyl-CoA of structural formula R—(C═O)-CoA, where R is a carbon chain of 5 carbons or fewer, 4 carbons or fewer, 3 carbons or fewer, or 2 carbons. In some embodiments, the carbon chain R comprises saturated and/or unsaturated carbon atoms. In some embodiments, the carbon chain R is a straight carbon chain. In some embodiments, the carbon chain R is a branched carbon chain. In some embodiments, the straight or branched carbon chain R is further substituted with a functional group, optionally wherein the function group is selected from —F, —Cl, —Br, —I, —NH₂, —OH.

In some embodiments, the present disclosure provides a recombinant or engineered thioesterase capable of hydrolyzing methacrylyl-CoA to methacrylic acid. Accordingly, the present disclosure also provides polynucleotides encoding such recombinant or engineered thioesterase capable of hydrolyzing methacrylyl-CoA to methacrylic acid, and vectors, and recombinant host cells comprising such polynucleotides. Further, the disclosure provides methods of using the recombinant host cells comprising polynucleotides encoding the recombinant or engineered thioesterase capable of hydrolyzing methacrylyl-CoA to methacrylic acid in a process for the production of methacrylic acid.

In some embodiments, the present disclosure provides a recombinant or engineered thioesterase capable of hydrolyzing 3-hydroxypropionyl-CoA to 3-hydroxypropionic acid (3HPA). Accordingly, the present disclosure also provides polynucleotides encoding such recombinant or engineered thioesterase capable of hydrolyzing 3-hydroxypropionyl-CoA to 3HPA, and vectors, and recombinant host cells comprising such polynucleotides. Further, the disclosure provides methods of using the recombinant host cells comprising polynucleotides encoding the recombinant or engineered thioesterase capable of hydrolyzing 3-hydroxypropionyl-CoA to 3HPA in a process for the production of 3HPA.

In some embodiments, the recombinant microorganism will be engineered to further include one or more additional heterologous genes. For example, in some embodiments, the recombinant microorganism will contain one, two, three or four heterologous genes encoding different polypeptides. For example in one embodiment, the one or more heterologous genes code for other enzymes in the acrylic acid pathway for example a lactyl-CoA producing enzyme and/or an acrylyl-CoA producing enzyme. In some embodiments, the microorganism that produces the acrylic acid according to the invention and comprises a heterologous gene encoding a lactyl-CoA producing enzyme and/or an acrylyl-CoA producing enzyme will also include an endogenous lactyl-CoA producing enzyme and/or an endogenous acrylyl-CoA producing enzyme. In other embodiments, the recombinant microorganism may already comprise metabolic pathways that allow accumulation of desired intermediates such as lactic acid, lactyl-CoA, and/or acrylyl-CoA.

In addition to comprising a heterologous acrylyl-CoA hydrolase, recombinant microorganisms of the invention may be engineered to include the inactivation of certain genes. Gene inactivation or disruption refers to any genetic modification that decreases or eliminates the expression of the gene and/or the functional activity of the corresponding gene product (mRNA and/or protein). Genetic modifications include complete or partial inactivation, suppression, deletion, interruption, blockage, or down-regulation of a gene. This can be accomplished, for example, by gene “knockout” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. Methods known in the art may be used to achieve gene disruptions including methods available from GeneBridges (Dresden Germany) and Red ET recombination (U.S. Pat. Nos. 6,355,412 and 6,509,156). Additional methods are also disclosed in Methods in Yeast Genetics, D. Amberg et al., Cold Spring Harbor Press, 2005 Ed. One non-limiting example would be limiting the production of propionyl-CoA in a microbial cell by targeting the gene(s) responsible for conversion to propionyl-CoA s from various substrates including pyruvate or methylcitrate. Another non-limiting example would be limiting the production of β-alanyl-CoA in a microbial cell by targeting the genes responsible for conversion of β-alanine.

The present invention makes use of recombinant nucleic acid constructs comprising a sequence encoding an acrylyl-CoA hydrolase (such as a TE as described above). The nucleic acid constructs of the present invention comprise vectors, such as a plasmid, a cosmid, a phage, a bacterial artificial chromosome (BAC), a yeast artificial chromosome (YAC) and the like into which a polynucleotide according to the invention has been inserted. In a particular aspect the present invention provides an expression vector comprising a polynucleotide coding a TE polypeptide operably linked to a promoter. The promoter may be heterologous or homologous to the TE. Expression vectors of the present invention may be used to transform an appropriate host cell to permit the host to express the TE enzyme. Methods for recombinant expression of proteins in fungi and other organisms are well known in the art, and a number expression vectors are available or can be constructed using routine methods. See, e.g., Tkacz and Lange, 2004, ADVANCES IN FUNGAL BIOTECHNOLOGY FOR INDUSTRY, AGRICULTURE, AND MEDICINE, KLUWER ACADEMIC/PLENUM PUBLISHERS. New York; Zhu et al., 2009, Construction of two Gateway vectors for gene expression in fungi Plasmid 6:128-33; Kavanagh, K. 2005, FUNGI: BIOLOGY AND APPLICATIONS Wiley, all of which are incorporated herein by reference. Large numbers of suitable vectors and promoters are known to those of skill in the art.

For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus megaterium promoters, and prokaryotic beta-lactamase gene (VIIIa-Kamaroff et al., Proc. Natl. Acad. Sci. USA 75: 3727-3731 (1978)), as well as the tac promoter (DeBoer et al., Proc. Natl. Acad. Sci. USA 80: 21-25 (1993)). Additional promoters include trp promoter, phage lambda PL, T7 promoter, promoters found at PromEC and the like. Promoters suitable for use in the present disclosure are described in Terpe, H., 2006, Appl. Microbiol. Biotecnol. 72:211-222.

In various embodiments, the DNA constructs and vectors comprising polynucleotides encoding a heterologous polypeptide are suitable for expression in yeast. In certain embodiments the promoter is a Y. lipolytica promoter. For yeast host cells, suitable promoters for directing transcription of the nucleic acid constructs of the present disclosure are known to the skilled artisan and include, but are not limited to, an enolase (ENO-1_gene) promoter, a galactokinase (GAL1) promoter, an alcohol dehydrogenase/glyceraldehyde-3-phosphate dehydrogenase (ADH2/GAP) promoter, a translation elongation factor EF-1 alpha (TEF1) promoter as well as those described by Romanos et al. (1992) Yeast 8:423-488. In other embodiments, promoters include the TEF1 promoter and an RPS7 promoter.

Examples of suitable promoters useful for directing the transcription of the nucleotide constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nunberg et al., 1984, Mol. Cell Biol., 4:2306-2315, Boel et al., 1984, EMBO J. 3:1581-85 and EPA 137280, all of which are incorporated herein by reference), and mutant, truncated, and hybrid promoters thereof. In a yeast host, useful promoters can be from the genes for Saccharomyces cerevisiae enolase (eno-1), Saccharomyces cerevisiae galactokinase (gal1), Saccharomyces cerevisiae alcohol dehydrogenase/glyceraldehyde-3-phosphate dehydrogenase (ADH2/GAP), and S. cerevisiae 3-phosphoglycerate kinase. Other useful promoters for yeast host cells are described by Romanos et al., 1992, Yeast 8:423-488, incorporated herein by reference. Promoters associated with chitinase production in fungi may be used. See, e.g., Blaiseau and Lafay, 1992, Gene 120243-248 (filamentous fungus Aphanocladium album); Limon et al., 1995, Curr. Genet, 28:478-83 (Trichoderma harzianum), both of which are incorporated herein by reference. Additional promoters include those from M. thermophila, provided in U.S. Prov. Patent Appln. Ser. Nos. 61/375,702, 61/375,745, 61/375,753, 61/375,755, and 61/375,760, all of which were filed on Aug. 20, 2010, and are hereby incorporated by reference in their entireties, as well as WO 2010/107303.

Any other promoter sequence that drives expression in a suitable host cell may be used. Suitable promoter sequences can be identified using well known methods. In one approach, a putative promoter sequence is linked 5′ to a sequence encoding a reporter protein, the construct is transfected into the host cell and the level of expression of the reporter is measured. Expression of the reporter can be determined by measuring, for example, mRNA levels of the reporter sequence, an enzymatic activity of the reporter protein, or the amount of reporter protein produced. For example, promoter activity may be determined by using the green fluorescent protein as coding sequence (Henriksen et al, 1999, Microbiology 145:729-34, incorporated herein by reference) or a lacZ reporter gene (Punt et al, 1997, Gene, 197:189-93, incorporated herein by reference). Functional promoters may be derived from naturally occurring promoter sequences by directed evolution methods. See, e.g. Wright et al., 2005, Human Gene Therapy, 16:881-892, incorporated herein by reference.

Cloned acrylyl-CoA hydrolases may also have a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary transcription terminators are described in U.S. Pat. No. 7,399,627, incorporated herein by reference.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-88.

A suitable leader sequence may be part of the heterologous sequence, which is a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

Sequences may also contain a polyadenylation sequence, which is a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-gludosidase. Useful polyadenylation sequences for yeast host cells are described by Guo and Sherman, Mol Cell Bio 15:5983-5990 (1995).

The expression vector of the present invention optionally contains one or more selectable markers, which permit easy selection of transformed cells. A selectable marker is a gene, the product of which provides for biocide or viral resistance, resistance to heavy metals, prototrophy to auxotrophs, and the like. Selectable markers for use in a filamentous fungal host cell include, but are not limited to, AmdS (acetamidase), ArgB (ornithine carbamoyltransferase), Bar (phosphinothricin acetyltransferase), Hph (hygromycin phosphotransferase), NiaD (nitrate reductase), PyrG (orotidine-5′-phosphate decarboxylase), CysC (sulfate adenyltransferase), and TrpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Suitable markers for yeast host cells are ADE2, HIS3, LEU2, LYS2, MET3, TRP1, and URA3.

Heterologous polynucleotide sequences including a polynucleotide sequence encoding an acrylyl-CoA hydrolase (such as a TE) can be introduced into a host microorganism using techniques well known in the art. Some of these techniques include but are not limited to electroporation, transduction, transfection, and the like (collectively referred to as transformation). The transformation of heterologous nucleic acid sequences such as a construct comprising a heterologous TE sequence can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the heterologous nucleic acid is expressed in a sufficient amount to produce the desired product, and it is further understood that expression levels can be optimized to obtain sufficient expression using methods well known in the art and as disclosed herein.

In some embodiments, the formation of the acrylyl-CoA substrate in a non-naturally occurring microorganism according to the invention is produced in a cell by the conversion of any one of the following compounds propanoyl CoA; lactoyl-CoA; β-alanyl CoA or 3-HP CoA (3-HP; 3-hydroxypropanoate).

### 6.4 Methods for Producing Acrylic Acid and Other Carboxylic Acids

This section of the present disclosure provides embodiments in the context of the specific conversion of acrylyl-CoA to acrylic acid, however it is intended (and the ordinary artisan will understand) that any of these embodiments may be implemented for the biocatalytic production of other carboxylic acid compounds disclosed herein (e.g., methacrylic acid, or 3HPA) from the corresponding acyl-CoA using the corresponding transformed recombinant microorganisms and/or acyl-CoA hydrolase enzymes.

In some embodiments, the conversion of acrylyl-CoA to acrylic acid may be carried out in vitro by contacting the acrylyl-CoA substrate with an acrylyl-CoA hydrolase (such as a TE) under suitable conditions of temperature, pH, and ionic strength and time sufficient for the production of acrylic acid. In some embodiments, the acrylic acid is produced in cell-free systems and the TE is provided in a partially or substantially pure form.

In some embodiments, the invention relates to a method of making acrylic acid comprising contacting an isolated acrylyl-CoA hydrolase (such as a TE) according to the invention in a culture medium including the substrate acrylyl-CoA under suitable conditions of temperature, time, pH and ionic strength for the conversion of acrylyl-CoA to acrylic acid. The culture medium may comprise a spent broth, a broth that no longer supports microbial growth or with limited capacity to support microbial growth or a broth which does support microbial growth. In this embodiment, the substrate acrylyl-CoA may be provided by production in a microbial cell, such as a cell described hereinabove.

In other embodiments, the method of producing an acrylic acid composition comprises culturing a recombinant (non-naturally occurring) microorganism (for example, but not limited to a Bacillus, a Lactobacillus, an Escherichia, a Rhizopus, an Issatchenkia, a Kluyveromyces, a Myceliophthora, a Rhodococcus, a Trichoderma, an Aspergillus, a Saccharomyces, a Pichia, a Candida, or a Yarrowia) in a suitable culture medium, wherein the recombinant microorganism comprises a gene encoding an acrylyl-CoA hydrolase (such as a TE) polypeptide as described above, allowing expression of said gene, wherein said expression results in the production of acrylic acid.

Fermentation or culturing of the recombinant microorganism is carried out under suitable conditions and for a time sufficient for production of acrylic acid. Conditions for the culture and production of cells, including filamentous fungi, bacterial and yeast cells, are readily available. Cell culture media in general are set forth in Atlas and Parks, Eds., The Handbook of Microbiological Media (1993) CRC Press, Boca Raton, Fla., which is incorporated herein by reference. The individual components of such media are available from commercial sources, e.g., under the Difco™ and BBL™ trademarks. In one non-limiting example, the aqueous nutrient medium is a “rich medium” comprising complex sources of nitrogen, salts, and carbon, such as YP medium, comprising 10 g/L of peptone and 10 g/L yeast extract of such a medium. In other non-limiting embodiments, the aqueous nutrient medium comprises a mixture of Yeast Nitrogen Base (Difco™) in combination supplemented with an appropriate mixture of amino acids, e.g., SC medium. In particular aspects of this embodiment, the amino acid mixture lacks one or more amino acids, thereby imposing selective pressure for maintenance of an expression vector within the recombinant host cell.

The recombinant microorganisms can be grown under batch or continuous fermentation conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, fermentations are carried out at temperatures within the range of from about 10° C. to about 60° C., from about 15° C. to about 50° C., from about 20° C. to about 45° C., from about 20° C. to about 40° C., from about 20° C. to about 35° C., and from about 25° C. to about 45° C. In a particular aspect, the fermentation is carried out at a temperature of from about 28° C. and also from about 30° C. In other embodiments, the fermentation is carried out for a period of time within the range of from about 4 hours to about 240 hours, from about 8 hours to about 240 hours, from about 8 hours to about 168 hours, from about 8 hours to about 144 hours, from about 16 hours to about 120 hours, or from about 24 hours to about 72 hours. In other embodiments, the fermentation will be carried out at a pH in the range of 3 to 8, in the range of 3 to 7, in the range of 4 to 7, in the range of 3 to 5 and also in the range of 4 to 5.5. In some preferred embodiments, the recombinant microorganism of the invention which is capable of producing acrylic acid will grow and produce acrylic acid under acidic pH conditions such as below pH 5.0, below pH 4.5, below pH4.0, and below pH 3.5.

Carbon sources useful in the aqueous fermentation medium or broth of the disclosed process in which the recombinant microorganisms are grown are those assimilable by the recombinant host strain. Assimilable carbon sources are available in many forms and include renewable carbon sources and the cellulosic and starch feedstock substrates obtained therefrom. Such examples include, for example, depolymerized cellulosic material, monosaccharides, disaccharides, oligosaccharides, saturated and unsaturated fatty acids, succinate, acetate and mixtures thereof. Further carbon sources include, without limitation, glucose, galactose, sucrose, xylose, fructose, glycerol, arabinose, mannose, raffinose, lactose, maltose, and mixtures thereof. “Fermentable sugars” refers to sugars (monosaccharides, disaccharides and short oligosaccharides) such as but not limited to glucose, xylose, galactose, arabinose, mannose and sucrose. Fermentable sugar is any sugar that a microorganism can utilize or ferment. In some embodiments, the term “fermentable sugars” is used interchangeably with the term “assimilable carbon source”.

In one aspect, fermentation is carried out with a mixture of glucose and galactose as the assimilable carbon source. In some preferred embodiments, the assimilable carbon source is from cellulosic and starch feedstock derived from but not limited to, wood, wood pulp, paper pulp, corn fiber, corn grain, corn cobs, crop residues such as corn husks, corn stover, grasses, wheat, wheat straw, barley, barley straw, hay, rice, rice straw, switchgrass, waste paper, paper and pulp processing waste, woody or herbaceous plants, fruit or vegetable pulp, corn cobs, distillers grain, grasses, rice hulls, cotton, hemp, flax, sisal, sugar cane bagasse, sorghum, soy, switchgrass, components obtained from milling of grains, trees, branches, roots, leaves, wood chips, sawdust, shrubs and bushes, vegetables, fruits, and flowers and any suitable mixtures thereof. In some embodiments, the cellulosic biomass comprises, but is not limited to cultivated crops (e.g., grasses, including C4 grasses, such as switch grass, cord grass, rye grass, miscanthus, reed canary grass, or any combination thereof), sugar processing residues, for example, but not limited to, bagasse (e.g., sugar cane bagasse, beet pulp [e.g., sugar beet], or a combination thereof), agricultural residues (e.g., soybean stover, corn stover, corn fiber, rice straw, sugar cane straw, rice, rice hulls, barley straw, corn cobs, wheat straw, canola straw, oat straw, oat hulls, corn fiber, hemp, flax, sisal, cotton, or any combination thereof), fruit pulp, vegetable pulp, distillers' grains, forestry biomass (e.g., wood, wood pulp, paper pulp, recycled wood pulp fiber, sawdust, hardwood, such as aspen wood, softwood, or a combination thereof). Furthermore, in some embodiments, the cellulosic biomass comprises cellulosic waste material and/or forestry waste materials, including but not limited to, paper and pulp processing waste, newsprint, cardboard and the like.

The acrylic acid maybe produced directly from the recombinant cells as described above or may be secreted from the cell. Further recovery of the acrylic acid may take place by standard separation and purification methods. For example acrylic acid and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art.

It is generally known in the art that acrylic acid may be toxic to cells. Therefore, in one embodiment an appropriate microorganism may be selected or engineered to withstand tolerance to acrylic acid. In general the recombinant cells should be tolerant to the presence of acrylic acid at levels up to at least 0.5%, at least 1%, at least 2%, at least 3%, at least 4%, at least 5%, at least 6%, at least 7%, at least 8%, at least 9%, at least 10% final titers of acrylic acid.

In some embodiments, production of acrylic acid in the culture or fermentation media should be possible to about 1 g/L, about 3 g/L, about 5 g/L; about 10 g/L, about 15 g/L, about 20 g/L, about 25 g/L, about 30 g/L, about 35 g/L, about 40 g/L about 45 g/L, about 50 g/L, about 60 g/L, about 70 g/L, about 80 g/L, about 90 g/L and even about 100 g/L or higher (Straathof et al., (2005) Appl. Microbiol. Biotechnol. 67: 727-734).

In some non-limiting preferred embodiments, the method comprises producing acrylic acid in a recombinant E. coli comprising introducing into the E. coli cell a polynucleotide encoding a thioesterase which is capable of converting acrylyl-CoA to acrylic acid and culturing the E. coli under sufficient culture conditions in the presence of a carbon source. In some embodiments, the thioesterase is a TE6 thioesterase and in some embodiments the recombinant E. coli will include at least 1, at least 2, at least 3 inactivated genes. In other embodiments the E. coli will include at least 1, at least 2, at least 3 additional heterologous genes such as but not limited to a lactyl-CoA producing enzyme and/or an acrylyl-CoA producing enzyme. In some embodiments the carbon source is a fermentable sugar such as but not limited to glucose that is obtained from a biomass source such as but not limited to corn stover, corn grain, wheat grass, or sugar cane bagasse. In some embodiments, at least 1 g/L of acrylic acid is produced and in some embodiments the acrylic acid is recovered from the culture.

In other embodiments, a lactic acid producing microorganism useful according to the methods of the invention will produce at least 5 g/L, at least 10 g/L, at least 30 g/L, at least 40 g/L, least 50 g/L, at least 60 g/L, at least 70 g/L at least 80 g/L, at least 90 g/L and at least 100 g/L or more of lactic acid as determined for example with a Bio-Rad Aminex HPX-87H column using standard HPLC methods.

In yet other embodiments, the recombinant microorganism comprising a heterologous acrylyl-CoA hydrolase (such as a TE) according to the invention may produce at least about 1 gram (g) of acrylic acid for every 100 grams (g) of glucose consumed; at least 5 g of acrylic acid for every 100 g of glucose consumed; at least 10 g of acrylic acid for every 100 g of glucose consumed; at least 20 g of acrylic acid for every 100 g of glucose consumed; at least 25 g of acrylic acid for every 100 g of glucose consumed; at least 30 g of acrylic acid or every 100 g glucose consumed, and at least 40 g of acrylic acid for every 100 g of glucose consumed in the culturing step.

The acrylic acid produced according to the invention may be further converted to various other useful compounds including but not limited to acrylic acid derivatives such as esters, salts, and amides. Esters include such derivatives as methyl acrylate, ethyl acrylate, n-butyl acrylate, hydroxypropyl acrylate, hydroxyethyl acrylate, isobutyl acrylate, and ethylhexyl acrylate. Amides include such derivatives as dimethylacrylamides and isopropylacrylamides. Salts include such derivatives as sodium acrylate, potassium acrylate and ammonium acrylate. Additional derivatives include compounds such as polyacrylic acid. Acrylic acid derivatives such as esters and polymers may be formed by standard methods including esterification and/or polymerization. For example a number of publications disclose the preparation of acrylic acid esters by reactions with lipase enzymes (U.S. Pat. No. 5,541,093). Reference is also made to U.S. Pat. No. 7,901,915 which includes a numbers of tests for lipase activity.

The following examples are for illustrative purposes and are not intended to limit the scope of the present invention.

### EXAMPLES

### Example 1

### Identification of Enzymes Displaying Acrylyl-CoA Hydrolase Activity

A. Gene Acquisition

Wild-type genes displaying acrylyl-CoA hydrolase activities from a wide range of organisms were designed for expression in E. coli based on reported amino acid sequences (See Table 2). All genes were codon optimized for expression in E. coli. Genes were synthesized by Genscript (Piscataway, N.J.) with flanking restriction sites for cloning into E. coli vector pCK110900.

The nucleotide sequence of SEQ ID NO: 7 was inserted immediately upstream of the ATG start codon to add XbaI, SfiI and SpeI restriction sites.

(SEQ ID NO: 7)
5′ ACAATCTAGAGGCCAGCCTGGCCATAAGGAGATACTAGT 3′

The nucleotide sequence of SEQ ID NO: 8 was inserted immediately preceding the TAG stop codon in order to add NgoMIV and SfiI restriction sites as well as six codons encoding for a hexahistidine tag.

(SEQ ID NO: 8.)
5′ GCCGGCGGCCAAACTGGCCACCATCACCATCACCAT 3′

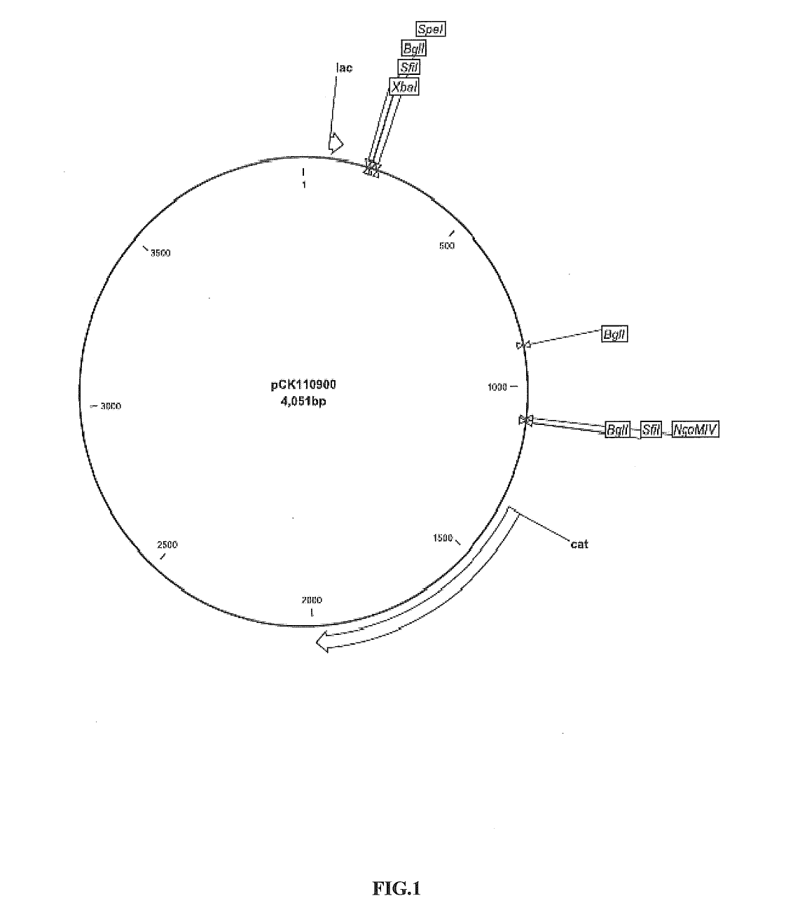

The plasmid of FIG. 1 illustrates the locations of the various promoters, genes, and restriction sites used. The genes sequences were verified by DNA sequencing.

TABLE 2

| | | | GenBank |
|---|---|---|---|
| | | Enzyme | Accession |
| Name¹ | Source Organism | Family² | number |
| Acot4 | Homo sapien | TE2 | NP_689544.3 |
| Pte-1/Tes1 | Saccharomyces cerevisiae | TE4 | DAA08810.1 |
| ScoT | Streptomyces coelicolor | TE4 | NP_630385.1 |
| | A3(2) | | |
| Pte-2 | Mus musculus | TE4 | AAL35333.1 |
| Acot8 | Rattus norvegicus | TE4 | EDL96495.1 |
| Cj0915 | Campylobacter jejuni | TE6 | YP_002344313.1 |
| YciA | Haemophilus influenza | TE6 | NP_438987.1 |
| | (HIO827) | | |
| YciA | Escherichia coli | TE6 | AAN80186.1 |
| Acot12 | Rattus norvegicus | TE6 | EDM10006.1 |
| | Deinococcus geothermalis | TE6 | YP_605627.1 |
| | Picrophilus torridus | TE6 | YP_023571.1 |
| | DSM 9790 | | |
| ACIAD3139 | Acinetobacter sp. ADP1 | TE6 | YP_047652.1 |
| | | | GI: 50086142 |
| Them2 | Homo sapien | TE8 | 2F0X |
| | Dictyostelium discoideum | TE8 | XP_636363 |
| YbgC | Helicobacter pylori | TE9 | 2PZH |
| 4-HBA-CoA | Pseudomonas sp. CBS3 | TE10 | 1lo8 |
| BH1999 | Bacillus halodurans C-125 | TE10 | BAB05718.1 |
| 4HBT | Arthrobacter sp | TE11 | 1Q4T |
| | Conexibacter woesei | TE11 | YP_003395277 |
| | DSM 1468 | | |
| PaaI | Azoarcus evansii | TE13 | AAG28967.1 |
| PaaI | Thermus thermophilus | TE13 | BAD70788.1 |
| | Methylibium petroleiphilum | TE13 | YP_001020182 |
| | PM1 | | |
| RifR | Amycolatopsis mediterranei | TE18 | AAG52991.1 |
| Tylosin TEII | Streptomyces fradiae | TE18 | AAA21345.1 |
| SrfD | Bacillus subtilis | TE18 | AAF87217.1 |
| | ATCC 21332 | | |
| | Rattus norvegicus | TE18 | P08635 |
| GrsT | Aneurinibacillus migulanus | TE18 | AAA58717 |
| Rv0098 | Mycobacterium | TE24 | 2PFC |
| | tuberculosis | | |
| | Streptomyces avermitilis | TE24 | NP_821781 |
| | MA-4680 | | |
| Flk | Streptomyces cattleya | TE25 | 3KX8 |
| DmdD | Ruegeria pomeroyi DSS-3 | Hydratase | YP_168993.1 |

¹Unnamed proteins are left blank.
²Families based on Cantu, Chen, Lemons & Reilly, (2010), PMID: 21045059, “ThYme: a database for thioester-active enzymes” Nucleic Acids Res. 39 (Database issue): D342-6..

B. Expression of Acrylyl-CoA Hydrolases (e.g., Thioesterases) in E. coli

Genes encoding thioesterases under control of a lac promoter were cloned into pCK110900 vector containing a P15a origin of replication and a chloramphenicol resistance gene (cat) used for replication and as a selective marker respectively. Chemically competent E. coli (W3110) cells were prepared by growing cells to an OD₆₀₀ of 0.4. The cells were centrifuged for 10 minutes at 1000×g, 4° C. and resuspended in 10% of the original culture volume with Transformation and Storage Solution (TSS) (20 mM MgCl₂, 5% DMSO and 10% PEG 8000 in Luria Broth (LB)). The cells were incubated on ice for 10 minutes and then aliquoted in 20 μl volumes for transformation. The resulting plasmids were transformed into E. coli (W3110) using heat shock (Yoshida, N. and Sato, M. 2009, Plasmid uptake by bacteria: a comparison of methods and efficiencies. Applied Microbiology and Biotechnology 83: 791-8). Transformed E. coli cells were selected by plating onto LB agar plates containing 1% glucose and 30 μg/ml chloramphenicol. After overnight incubation at 37° C., colonies were picked onto a NUNC 96-well shallow flat bottom plates filled with 180 μl/well LB supplemented with 1% glucose and 30 μg/ml chloramphenicol. Plates were allowed to grow overnight for 18-20 hours in a Kuhner shaker (200 rpm, 30° C., and 85% relative humidity). Overnight growth samples (20 μL) were transferred onto Costar 96-well deep plates filled with 380 uL of 2×YT broth (8 g/L tryptone, 5 g/L yeast extract and 5 g/L NaCl) supplemented with 30 μg/ml chloramphenicol and 0.4% glucose. Plates were incubated for 105 minutes in a Kuhner shaker (250 rpm, 30° C., and 85% relative humidity). Cells were then induced with 40 μL of 10 mM IPTG (isopropyl β-D-1-thiogalactopyranoside) in sterile water and incubated overnight for 20-24 hours in a Kuhner shaker (250 rpm, 30° C., and 85% relative humidity).

### Example 2

### Cell Lysis, Protein Purification, and Detection of CoA, Acrylyl CoA, and Acrylic Acid

E. coli overexpressing acrylyl-CoA hydrolases of interest as described above in Example 1 were centrifuged at 3500×g for 10 min. The supernatants were discarded and 200 μL aliquots of lysis buffer (50 mM HEPES, 100 mM KCl, 1.0 mM MgCl₂, 400 mM NaCl, 20 mM imidazole, pH 7.5), 0.5 mg/mL lysozyme, and 0.5 mg/mL Polymix B sulfate (PMBS)), were added to the cell pellets. Lysates were agitated at 220 rpm for 2 h at room temperature, and the lysis mixture was centrifuged at 3500×g for 10 min. Supernatants were loaded onto a GE Healthcare His SpinTrap FF plate pre-equilibrated with binding buffer (50 mM HEPES, 400 mM NaCl, 100 mM KCl, 20 mM imidazole, 1.0 mM MgCl₂pH 7.4), incubated for 5 min and then centrifuged at 100×g for 30 s. The column bound hydrolases were washed via equilibration with 400 uL binding buffer (30 s) and centrifugation (100×g 30 s), and eluted by addition of 200 μL of elution buffer (50 mM HEPES, 400 mM NaCl, 100 mM KCl, 500 mM imidazole, 1.0 mM MgCl₂ pH 7.4) and centrifugation (100×g 30 s). Biocatalytic cleavage of acrylyl-CoA to acrylic acid and CoA-SH (free form CoA with the SH group) was measured independently by colorimetric detection of the appearance of CoA-SH and by HPLC detection of the appearance of acrylic acid and disappearance of acrylyl-CoA.

Acrylyl-CoA was synthesized by a modification of the method described in U.S. Pat. No. 7,901,915, which is hereby incorporated by reference herein. Briefly, 5 mL of 0.2 M KHCO₃ and 50 mg of CoA (˜0.06 mmol) was added to a 6 mL preparatory HPLC vial under air and immersed in an ice bath. The resulting solution was stirred vigorously under air for 5 minutes. 50 μL of acrylyl chloride (0.6 mmol; ˜10 equiv.) was added to the reaction mixture and the resulting solution was stirred for 30 minutes in ice bath. The acrylyl-CoA was isolated by preparatory HPLC by a single injection of the entire reaction content onto a 21 mm diameter×(250 mm Gemini C18+Luna C18) column with a Luna guard cartridge. The compound was eluted at room temperature with a gradient of mobile phase A (25 mM, pH 7 ammonium formate) and mobile phase B (MeOH) running at 15 mL per minute. Gradient: 20% B→28% B in 16 minutes; 28% B→80% B in 1 minute; 80% B for 8 minutes. 5 mL fractions were collected every 20 seconds between 16 min and 20 min. UV analysis at 254 nm found the acrylyl-CoA typically eluted between 16 min and 20 min.

The identity of acrylyl-CoA was confirmed by LC/MS. Fractions with a signal above 2000 milli-absorbance units (mAu) were pooled (typically 8-10 fractions; ˜45 mL). 0.5 mL of 1.0 M (pH 7.0) potassium phosphate was added to the pooled fractions. The pooled fractions were then concentrated to approximately 5 mL by a rotator evaporator (˜30 mm Hg; 25° C.). The concentrated acrylyl-CoA solution was stored at −20° C. for no more than one week prior to use.

For detection of CoA-SH, purified hydrolases (5 μL) were added to a mixture of 25 μL of 2× reaction buffer (100 mM HEPES, 200 mM KCl, 2 mM MgCl₂, 0.8 mg/mL BSA, 1.0 mM 5,5′(dithiobis-(2-nitrobenzoic acid), pH 7.4) and 20 μL, of acrylyl-CoA solution (estimated at 150 μM based on UV absorbance). Samples were incubated at room temperature for 3 h and the release of CoA-SH was tracked by measuring the absorbance at 412 nM on a Molecular Devices Spectra Max Plus 384 UV/vis spectrophotometer.

Acrylyl-CoA hydrolases identified in Table 2 were synthesized, transformed, expressed and purified. Purified proteins were evaluated by SDS-PAGE and found to conform to the expected molecular weights. Release of CoA and % availability of acrylyl-CoA were evaluated. Hydrolase activity above the control, (a vector which did not include heterologous acrylyl-CoA hydrolase) was demonstrated rated. (Table 3).

TABLE 3

| | % of available |
|---|---|
| Acrylyl-CoA hydrolase | acrylyl-CoA hydrolyzed |
| Cj0915 [YP_002344313.1] | 8 |
| YP_023571.1 | 59 |
| ACIAD3139 [YP_047652.1; GI: 50086142] | 89 |
| (polypeptide of SEQ ID NO: 10) | |
| YciA [NP_438987.1] | 90 |
| YciA [AAN80186.1] | 100 |

Analysis of acrylic acid was performed by adding purified hydrolase (30 μL) to a mixture of 30 μL 4× activity buffer (200 mM HEPES, 400 mM KCl, 4.0 mM MgCl₂, pH 7.4) and 90 μL of acrylyl-CoA (˜150 uM). The sample (10 μL) was monitored by HPLC analysis on 25-cm×4.6 mm i.d. stainless steel column packed with Zorbax 8-Fm (Phenomene X) ODS-bound, spherical silica particles, eluting across a linear gradient of 18-40% (v/v) water acetonitrile containing 0.1% phosphoric acid at 1 mL/min. Acrylic acid was detected as a peak eluting at 4.4 minutes, absorbing at 230 nm. Analysis of acrylyl-CoA was performed by addition of 20 μL of hydrolase enzyme to a mixture of 65 μL of 4× activity buffer and 180 μL of acrylyl-CoA. Disappearance of acrylyl-CoA was measured by integrating the acrylyl-CoA peak isolated as essentially described in Example 1 of U.S. Pat. No. 7,901,915. The appearance of acrylic acid and disappearance of acrylyl-CoA was confirmed for AAN80186.1 using the method described herein.

### Example 3

### Preparation of Engineered Polypeptide Variants with Improved Activity and Selectivity for Acryloyl-CoA Derived from Wild-Type Thioesterase from Acinetobacter sp. ADP1 (YP_{—}047652)

Gene Synthesis and Optimization:

The polynucleotide sequence of SEQ ID NO: 1 encoding the wild-type thioesterase from Acinetobacter sp. ADP 1 polypeptide of SEQ ID NO: 2 (GenBank accession: YP_{—}047652.1; GI:50086142), was codon-optimized and modified with a His tag as described in Example 1, resulting the synthetic gene of SEQ ID NO: 9 which encodes the His tag modified thioesterase polypeptide of SEQ ID NO: 10. The synthetic gene of SEQ ID NO: 9 cloned into the E. coli W3110 expression construct under the control of the lac promoter as described in Example 1 was used as the starting gene for directed evolution of engineered thioesterase polypeptides having improved activity for acrylyl-CoA hydrolysis. To identify likely sites for improved activity and selectivity of the thioesterase, a homology model was built based on homologous (49% identity) H. influenze acyl-CoA thioesterase (1YLI) crystal structure with a CoA-SH ligand bound. To better approximate the structural consequence of a thioester, a hexanoyl-CoA substrate was docked into this model based on the Thermus thermophilus thioesterase (1WN3) crystal structure Amino acids within 7 Å of the thioester sulfur atom in the hexanoyl-CoA model or within 6 Å of the terminal CoA-SH sulfur atom in the CoA only model were targeted for mutation in the first round of evolution. Directed evolution of the codon-optimized thioesterase gene was carried out by constructing libraries of variant genes in which these positions associated with certain structural features were subjected to mutagenesis. These libraries were then plated, grown-up, and screened using high-throughput (HTP) assays as described below to provide a first round (“Round 1”) of 13 engineered thioesterase variant polypeptides with improved acrylyl-CoA hydrolysis activity relative to the His-tag modified “wild-type” thioesterase polypeptide of SEQ ID NO: 10. As shown in Table 4, these 13 improved Round 1 variants (having even numbered sequence identifiers SEQ ID NO: 12-36) each has an amino acid difference relative to SEQ ID NO: 10 at one of the following positions 34, 40, 54, 55, 66, 68, and 117. Due to its 2.71 fold-improvement-over-parent (FIOP) polypeptide of SEQ ID NO: 10 in acrylyl-CoA activity, the Round 1 evolved variant polypeptide of SEQ ID NO: 24, which has the A55V amino acid difference, was used as the parent backbone polypeptide sequence for the second round of directed evolution. The amino acid differences identified in the other 12 Round 1 variants were recombined with the A55V amino acid difference to build Round 2 libraries. These Round 2 libraries were then screened with the acrylyl-CoA substrate for improved activity relative to the parent polypeptide of SEQ ID NO: 24. Round 2 of directed evolution resulted in the 19 engineered thioesterase polypeptides having the even numbered sequence identifiers of SEQ ID NO: 38-74. These Round 2 thioesterase polypeptide variants have from 2 to 6 amino acid differences relative to SEQ ID NO: 10 and have improved activity and selectivity for hydrolyzing the acrylyl-CoA substrate relative to the activity and selectivity of the His-tag modified “wild-type” polypeptide of SEQ ID NO: 10.

TABLE 4

| | | Activity | | | | |
|---|---|---|---|---|---|---|
| SEQ | | (relative to SEQ ID NO: 10) | | | Substrate Selectivity | |
| ID | Amino Acid Differences | | D- | L- | Acrylyl- | Acrylyl- |
| NO: | (relative to SEQ | Acrylyl- | lactoyl- | lactoyl- | vs | vs |
| (nt/aa) | ID NO: 10) | CoA ¹ | CoA ² | CoA ³ | D-lactoyl | L-lactoyl |
| 9/10 | none | 1 | 1 | 1 | 1 | 1 |
| 11/12 | I34T; | 1.23 | 0.99 | 1.05 | 1.25 | 1.17 |
| 13/14 | V68L; | 1.24 | 1.02 | 1.18 | 1.22 | 1.05 |
| 15/16 | L40M; | 1.29 | 1.03 | 0.91 | 1.25 | 1.42 |
| 17/18 | V117L; | 1.10 | 0.97 | 0.88 | 1.13 | 1.25 |
| 19/20 | V66I; | 0.90 | 1.00 | 0.86 | 0.90 | 1.05 |
| 21/22 | V68R; | 1.06 | 1.00 | 0.93 | 1.05 | 1.14 |
| 23/24 | A55V; | 2.71 | 1.01 | 1.21 | 2.68 | 2.25 |
| 25/26 | A55S; | 1.67 | 0.91 | 0.73 | 1.82 | 2.29 |
| 27/28 | C54V; | 1.05 | 1.02 | 0.74 | 1.03 | 1.43 |
| 29/30 | L40I; | 1.15 | 0.99 | 0.83 | 1.16 | 1.39 |
| 31/32 | L40V; | 1.02 | 1.01 | 0.80 | 1.01 | 1.27 |
| 33/34 | L40A; | 1.18 | 0.99 | 0.76 | 1.19 | 1.56 |
| 35/36 | C54A; | 0.91 | 0.99 | 0.79 | 0.92 | 1.15 |
| 37/38 | I34T; A55S; | 1.90 | 0.45 | n.d. | 4.25 | n.d. |
| 39/40 | A55V; I34T; L40A; V68L; | 2.45 | 0.45 | n.d. | 5.43 | n.d. |
| 41/42 | A55V; I34T; V68L; | 3.14 | 0.73 | n.d. | 4.31 | n.d. |
| | V117L; | | | | | |
| 43/44 | I34T; A55S; V66I; | 1.84 | 0.48 | n.d. | 3.86 | n.d. |
| 45/46 | A55V; I34T; L40V; C54V; | 2.45 | 0.66 | n.d. | 3.72 | n.d. |
| | V66I; V68L; | | | | | |
| 47/48 | A55V; I34T; V66I ;V68L; | 2.50 | 0.62 | n.d. | 4.03 | n.d. |
| 49/50 | A55V; I34T; L40M; V66I; | 1.93 | 0.42 | n.d. | 4.60 | n.d. |
| | V68L; | | | | | |
| 51/52 | A55V; I34T; L40A; V66I; | 1.41 | 0.27 | n.d. | 5.28 | n.d. |
| | V68L; | | | | | |
| 53/54 | A55V; L40A; C54A; | 2.71 | 0.55 | n.d. | 4.88 | n.d. |
| 55/56 | A55V; L40V; C54A; | 3.32 | 0.85 | n.d. | 3.92 | n.d. |
| | V68L; | | | | | |
| 57/58 | A55V; I34T; L40M; C54G; | 2.23 | 0.51 | n.d. | 4.40 | n.d. |
| | V66I; V68L; | | | | | |
| 59/60 | A55V; I34T; V66I; | 2.20 | 0.59 | n.d. | 3.75 | n.d. |
| 61/62 | A55V; L40V; C54A; V66I; | 2.54 | 0.67 | n.d. | 3.77 | n.d. |
| | V68L; | | | | | |
| 63/64 | L40A; C54A; A55S; V66I; | 2.76 | 0.66 | n.d. | 4.20 | n.d. |
| 65/66 | A55V; L40A; V66I; V68L; | 4.19 | 1.14 | n.d. | 3.66 | n.d. |
| 67/68 | A55V; I34T; V68L; | 3.24 | 0.77 | n.d. | 4.19 | n.d. |
| 69/70 | A55V; I34T; L40M; C54A; | 1.70 | 0.37 | n.d. | 4.54 | n.d. |
| | V66I; | | | | | |
| 71/72 | A55V; I34T; L40A; V66I; | 1.62 | 0.45 | n.d. | 3.62 | n.d. |
| | V117L; | | | | | |
| 73/74 | A55V; L40A; V661; V68R;4.85 | 1.14 | n.d. | 4.24 | n.d. | |

High-Throughput (HTP) Growth, Expression, and Lysate Preparation:

Transformed E. coli cells expressing the engineered thioesterase variant genes were grown and expressed as described in Example 1 for the cloned wild-type thioesterase genes. Preparation of lysates of the transformed E. coli expressing the engineered thioesterase variant genes for use in HTP assay of acyryl-CoA hydrolysis activity was as carried out as follows: E. coli overexpressing acrylyl-CoA hydrolases of interest as described above in Example 1 were centrifuged at 3500×g for 10 min. The supernatants were discarded and 400 μL aliquots of lysis buffer (50 mM HEPES, 100 mM KCl, 1.0 mM MgCl₂, 400 mM NaCl, pH 7.5), 0.5 mg/mL lysozyme, and 0.5 mg/mL Polymix B sulfate (PMBS)), were added to the cell pellets. Lysates were agitated at 220 rpm for 2 h at room temperature, and the lysis mixture was centrifuged at 3500×g for 10 min. Supernatants were diluted 1:200 in dilution buffer (50 mM HEPES, 100 mM KCl, 1.0 mM MgCl₂, pH 7.5).

HTP Screening Assays of Engineered Thioesterase Polypeptides:

High-throughput screening used to guide primary selection of variants was carried out in 96-well plates using cell lysate. Activity was determined by detection of CoA-SH. Diluted lysates (25 μL) were added to a mixture of 25 μL of 4× reaction buffer (200 mM HEPES, 400 mM KCl, 1 mM MgCl₂, 1.6 mg/mL BSA, 4.0 mM 5,5′(dithiobis-(2-nitrobenzoic acid), pH 7.4) and 50 μL of CoA-ester solution (Acrylyl-CoA, D-lactoyl-CoA, or L-lactoyl-CoA estimated at between 250-1000 μM based on UV absorbance). Samples were incubated at room temperature for 20 min and the release of CoA-SH was tracked by measuring the absorbance at 412 nM on a Molecular Devices Spectra Max Plus 384 UV/vis spectrophotometer.

Synthesis of D- and L-Lactoyl-CoA:

To a 250 mL flask under air was added sequentially, 150 mL of 0.1 M Tris-HCl buffer, pH 7.5, 4.0 g of sodium lactate (d- or l-) (Sigma-Aldrich), 6.0 mL of 1 M MgCl₂, 1.6 g of ATP (sodium salt) (Sigma-Aldrich), and 400 mg of co-enzyme A (tri-lithium salt) (Oriental Yeast) to give a colorless solution (pH ˜5.2). The pH was adjusted to 7.4 via drop-wise addition of 50 wt % NaOH. To the pH adjusted solution was added 100 mg of S-acetyl-CoA synthetase from Baker's yeast (Sigma-Aldrich) to give a slightly cloudy solution. After stirring at room temperature for 16 hours, 160 mL of acetonitrile was added to give a milky mixture. After centrifugation at 3200 rcf at 20° C. for 10 minutes, the clear supernatant was decanted and concentrated via rotatory evaporator (˜30 mm Hg; 30° C.) until ˜50 mL remained. The product was purified via preparatory HPLC (5 mL per injection) using the instrumental parameters and conditions shown in Table 5.

Typically, lactoyl-CoA (D- or L-) eluted between 23 and 25 min. The pooled fractions were stabilized by addition of 0.5 mL of 1 M pH 7 potassium phosphate. The identity of lactoyl-CoA was confirmed by LC/MS.

TABLE 5
Column:
21 mm diameter × (250 mm Gemini C18 + Luna C18) with Luna guard
cartridge
Mobile phase A:
25 mM ammonium formate, pH 7
Mobile Phase B:
MeOH
Gradient:
5% B → 30% B in 25 minutes;
30% B → 80% B in 1 minute;
80% B for 4 minutes.
Mobile phase flow rate: 15 mL/min
Fractions collected every 20 seconds (5 mL) between t = 22 and 26 min.
Column at room temperature;
Detection at 254 nm

### Example 4

### Transformation and Growth of Yeast Strains

The genes coding for acrylyl-CoA hydrolases are synthesized with a codon bias for expression in S. cerevisiae. Ligation of these polynucleotides into a yeast expression vector PLS 1565 is performed using BamHI and NdeI restriction sites using standard procedures and protocols (see e.g., Sambrook et al., MOLECULAR CLONING: A LABORATORY MANUAL, 3rd Ed., Cold Spring Harbor Laboratory, NY (2001) and CURRENT PROTOCOLS IN MOLECULAR BIOLOGY, F. M. Ausubel et al., eds., Current Protocols (as supplemented through 2009), placing the genes under control of the TEF1 promoter. The plasmid PLS 1565 contains the TEF1 promoter for gene expression, KanMX resistance marker for antibiotic selection in S. cerevisiae, CEN4 and ARSH4 sequences for plasmid replication (Sikorski, R. S., and Hieter, P., 1989, “A system of shuttle vectors and yeast host strains designed for efficient manipulation of DNA in Saccharomyces cerevisiae,” Genetics 122: 19-27), and an E. coli plasmid replication origin with the ampicillin resistance marker for antibiotic selection in E. coli.

The resulting plasmids containing the various genes encoding for acrylyl-CoA hydrolases are used to transform S. cerevisiae strain NRRL YB-1952 using the lithium acetate, polyethylene glycol, and single-stranded carrier DNA method (Gietz and Woods, METHODS IN MICROBIOLOGY, vol. 26, chapter 4, Academic Press Ltd, 1998). Yeast cells are pre-cultured in YPD liquid medium (Difco YPD Broth containing 10 g/L yeast extract, 20 g/L peptone and 20 g/L dextrose), incubated at 30° C. and 250 rpm for 18 hours. Growth is monitored by measuring the optical density at 600 nm. Fresh YPD liquid medium is inoculated with sufficient cells from the pre-culture to obtain a starting optical density of 0.5. After approximately 2 to 3 hours of growth at 30° C. and 250 rpm, an optical density of approximately 1.2 is obtained. Cells are pelleted and resuspended in 0.5 mL of water. For each transformation, 100 ng to 500 ng of purified plasmid DNA is added to 50 μL of yeast cells. A mixture of 1000 μL of 50% PEG3350, 150 μL of 1 M lithium acetate, and 36 μL of single stranded salmon sperm DNA is added. The mixture is incubated at 30° C. for 10 minutes followed by 42° C. for 15 minutes. Cells are pelleted by centrifugation for 5 seconds and resuspended in 1 mL of fresh YPD liquid medium and grown at 30° C. for 2 hours. Recovered cells are plated on YPD agar medium supplemented with 200 μg/mL G-418 antibiotic for selection and incubated for 48 h at 30° C. Colonies are picked onto a NUNC 96-well shallow flat bottom plates filled with 180 μl/well YPD liquid medium supplemented with 200 μg/ml G-418. Plates are grown in a Kuhner shaker (200 rpm, 30° C., and 85% relative humidity).

All references cited herein including patents, published patent applications, papers and text book are hereby incorporated by reference in their entirety. The foregoing description and examples detail certain preferred embodiments of the invention. It will be appreciated that the invention may be practiced in many ways and the invention should be construed in accordance with the appended claims and any equivalents thereof.

1. A non-naturally occurring microorganism comprising:
(a) a pathway that produces an acyl-CoA compound of formula R—(C═O)-CoA, wherein R is a carbon chain of 5 carbons or fewer; and
(b) a heterologous polynucleotide encoding an acyl-CoA hydrolase capable of catalyzing the hydrolysis of the acyl-CoA compound, R—(C═O)-CoA, to the carboxylic acid compound, R—CO₂H.

2. The microorganism of claim 1, wherein the acyl-CoA compound of formula R—(C═O)-CoA is selected from: acrylyl-CoA, methacrylyl-CoA, and 3-hydroxypropionyl-CoA.

3. The microorganism of claim 1, wherein the carboxylic acid compound, R—CO₂H is selected from: acrylic acid, methacrylic acid, and 3-hydroxypropionic acid (3HPA).

4. The microorganism of claim 1, wherein the acyl-CoA hydrolase encoded by the heterologous polynucleotide is a thioesterase, optionally wherein the thioesterase is classified as a TE6 thioesterase, and optionally is derived from one of the following genes: Campylobacter jejuni (YP_{—}002344313.1); Haemophilus influenza (HIO827)(NP_{—}438987.1); Escherichia coli (AAN80186.1); Rattus norvegicus (EDM10006.1); Deinococcus geothermalis (YP_{—}605627.1); Picrophilus torridus DSM 9790 (YP_{—}023571.1); and Acinetobacter sp. ADP1 (YP_{—}047652.1, GI:50086142).

5. The microorganism of claim 1, wherein the acyl-CoA hydrolase is a thioesterase comprising an amino acid sequence having at least 80% identity to a sequence selected from SEQ ID NO: 2, 4, 6, and 10.

6. The microorganism of claim 1, wherein the acyl-CoA hydrolase is an engineered thioesterase which comprises an amino acid sequence having at least 80% identity to a sequence selected from SEQ ID NO: 2, 10, or even numbered SEQ ID NO: 12-74 and comprises one or more amino acid residue differences as compared to SEQ ID NO:2 or 10 at residue positions selected from: I34, L40, C54, A55, V66, V68, and V117, and optionally wherein the amino acid differences are selected from I34T, L40A, L40I, L40M, L40V, C54A, C54V, A55S, A55V, V66I, V68L, V68R, and V117L.

7-8. (canceled)

9. The microorganism of claim 1, wherein the acyl-CoA hydrolase is an engineered thioesterase capable of hydrolyzing acrylyl-CoA to acrylic acid, optionally wherein the thioesterase has an activity for hydrolyzing acrylyl-CoA to acrylic acid that is at least 1.5-fold greater than the activity of the thioesterase of SEQ ID NO: 10.

10. The microorganism of claim 1, wherein the microorganism is selected from the group consisting of yeast, bacteria, and filamentous fungi.

11. (canceled)

12. The microorganism of claim 1, wherein the microorganism is a lactic acid producing microorganism.

13. The microorganism of claim 1, wherein the microorganism further comprises one or more heterologous genes of an acrylic acid pathway, optionally wherein the heterologous genes comprising encoding a lactyl-CoA producing enzyme and/or an acrylyl-CoA producing enzyme.

14. The microorganism of claim 1, wherein the microorganism further comprises one or more gene disruptions that confer increased production of the carboxylic acid compound, R—CO₂H on the transformed microorganism.

15. A method for making carboxylic acid compound, R—CO₂H, wherein R is a carbon chain of 5 carbons or fewer, said comprising
a) providing a microorganism of claim 1, and
b) culturing the microorganism under sufficient culture conditions in the presence of a carbon source to promote the expression of the acyl-CoA hydrolase and production of the carboxylic acid compound.

16. The method of claim 15, wherein the carboxylic acid compound is selected from: acrylic acid, methacrylic acid, and 3-hydroxypropionic acid (3HPA).

17. The method of claim 15, wherein the carbon source comprises glucose, sucrose or combinations thereof, and/or the carbon source is derived from cellulosic biomass.

18. (canceled)

19. The method of claim 15, wherein the carboxylic acid compound is produced in an amount of at least 1 g/L or about 5 g/L of culture media.

20. (canceled)

21. The method of claim 15, wherein the method further comprises recovering the produced carboxylic acid compound.

22. The method of claim 21, wherein the carboxylic acid compound is acrylic acid and the method further comprises modifying the produced and recovered acrylic acid to a salt, an amide, an ester derivative of acrylic acid or a polyacrylic acid.

23. (canceled)

24. A method for producing acrylic acid comprising: (a) transforming a lactic acid producing microorganism with a heterologous polynucleotide encoding a thioesterase polypeptide, wherein the thioesterase polypeptide is capable of converting acrylyl CoA to acrylic acid; (b) culturing the transformed lactic acid producing microorganism in the presence of a carbon source and under sufficient conditions to produce acrylic acid; and (c) recovering the acrylic acid.

25. The method according to claim 24 further comprising transforming the microorganism with at least one additional polynucleotide encoding a lactyl-CoA producing enzyme and/or an acrylyl-CoA producing enzyme.

26. A method for hydrolyzing acrylyl-CoA to acrylic acid or a derivative thereof comprising contacting an effective amount of a thioesterase (TE) with an acrylyl-CoA substrate for a period of time and under sufficient culture conditions to produce acrylic acid, wherein the TE is characterized by its ability to hydrolyze acrylyl-CoA to acrylic acid and wherein the acrylyl-CoA and/or the TE is produced from a cultured microbial cell, and wherein the TE has an amino acid sequence with at least 90%, 91%, 92%, 93%, 94%, 95%, 96%, 97%, 98%, or 99% sequence identity to SEQ ID NO:2, 4, 6, or 10.

27-28. (canceled)

29. The method of claim 26, wherein the thioesterase comprises an amino acid sequence having at least 80% identity to a sequence selected from SEQ ID NO: 2, 10, or even numbered SEQ ID NO: 12-74 and comprises one or more amino acid residue differences as compared to SEQ ID NO:2 or 10 at residue positions selected from: I34, L40, C54, A55, V66, V68, and V117, and optionally wherein the amino acid differences are selected from I34T, L40A, L40I, L40M, L40V, C54A, C54V, A55S, A55V, V66I, V68L, V68R, and V117L.

30. (canceled)

31. The method of claim 26, wherein the method further comprises separating the acrylic acid from the culture.

32. The method of claim 31, wherein the method further comprises modifying the acrylic acid to a salt, an amide or an ester derivative.

33. A method for making acrylic acid comprising reacting acrylyl-CoA in the presence of water and an acrylyl-CoA hydrolase to produce acrylic acid.

34. The method of claim 33, wherein the method is conducted in vitro, in vivo, or partially in vitro and partially in vivo.

35-36. (canceled)

37. The method of claim 33, wherein the acrylyl-CoA hydrolase is a thioesterase (TE).

38. The method of claim 33, wherein the TE has an amino acid sequence with at least 90%, 91%, 92%, 93%, 94%, 95%, 96%, 97%, 98%, or 99%, sequence identity to SEQ ID NO: 2, 4, 6, or 10.

39. The method of claim 33, wherein the TE comprises an amino acid sequence having at least 80% identity to a sequence selected from SEQ ID NO: 2, 10, or even numbered SEQ ID NO: 12-74, and comprises one or more amino acid residue differences as compared to SEQ ID NO:2 or 10 at residue positions selected from: I34, L40, C54, A55, V66, V68, and V117, and optionally wherein the amino acid differences are selected from I34T, L40A, L40I, L40M, L40V, C54A, C54V, A55S, A55V, V66I, V68L, V68R, and V117L.

40. (canceled)

41. An engineered thioesterase (TE) polypeptide which comprises an amino acid sequence having at least 80% identity to a sequence selected from SEQ ID NO: 2, 10, or the even numbered SEQ ID NO: 12-74, and comprises one or more amino acid residue differences as compared to SEQ ID NO:2 or 10 at residue positions selected from: I34, L40, C54, A55, V66, V68, and V117, and wherein said engineered thioesterase hydrolyzes acrylyl-CoA to acrylic acid with a hydrolytic activity level that is at least 1.5-fold greater than the hydrolytic activity of the thioesterase of SEQ ID NO:10.

42-45. (canceled)
